(19) United States
(12) Patent Application Publication
(10) Pub. No.: US 2010/0239493 A1
(43) Pub. Date: Sep. 23, 2010

(54) METHODS AND COMPOUNDS FOR FORMING MONOLITHIC TITANIA, OPTIONALLY BIOMOLECULE DOPED, WITH CONTROLLED MORPHOLOGY USING BIOCOMPATIBLE SOL-GEL PROCESSES

(75) Inventors: Yunyu Yi, Winnipeg (CA); Michael A. Brook, Ancaster (CA); John D. Brennan, Dundas (CA); Yang Chen, Hamilton (CA)

Correspondence Address: FULBRIGHT & JAWORSKI L.L.P., 600 CONGRESS AVE., SUITE 2400, AUSTIN, TX, 78701, US

(73) Assignee: MCMASTER UNIVERSITY, Hamilton, ON (CA)

(21) Appl. No.: 12/442,434

(22) PCT Filed: Sep. 24, 2007
(86) PCT No.: PCT/CA07/01693

§ 371 Date: May 19, 2010

Related U.S. Application Data
(60) Provisional application No. 60/826,662, filed on Sep. 22, 2006.

(51) Int. Cl. C01G 23/047; C07F 7/28; C07H 23/00

(52) U.S. Cl. 423/610; 556/51; 536/121

(57) ABSTRACT

The present invention includes polyol modified titanium compounds, their preparation and use in methods to make biomolecule compatible monolithic titania. The invention also includes the use of the biomolecule compatible monolithic titania in bioanalytic applications, for example in biosensors, chromatographic columns, microarrays and bioaffinity columns.

### FIELD OF THE INVENTION

The present invention relates to methods of preparing biomolecule compatible monolithic titania materials, to the titania materials prepared using these methods and to uses of the titania materials, in particular as chromatographic supports, biosensors and/or to immobilize enzymes.

### BACKGROUND OF THE INVENTION

### (a) Silica Chromatography

Silica in a variety of particulate forms has been extensively utilized as chromatographic support. Partition of dissolved molecules between the hydrophilic siliceous surface and a flowing solvent permits the separation of compounds on many different scales (ng→kg scales). The efficiency of separation in these systems is related to the surface area of the silica to which the compound mixture is exposed.

There is an important physical limitation to practical separation with packed particulate systems. As the number of theoretical plates increases there is an attendant increase in backpressure on the column. There is, therefore, a trade off between higher separation efficiency and practical operating pressures. High pressures have attendant danger, and/or are impractical from the perspective of cost. Even with highly efficient columns operating at high pressures, the throughput that can be realized is often relatively low.¹

Significant improvement in the surface area/back pressure relationship can be realized by the use of self-supporting monolithic silica columns.^{1,2} For example, styrene monoliths have been reported to be useful for polynucleotide separation.³ The group of Tanaka, in particular, have reported the preparation of silica monoliths.⁴ Merck currently sells monolithic silica columns under the Chromolith™ label.⁵ The structure of these monoliths involves a series of distorted silica spheres fused by a layer of silica. The presence of macropores, between linked silica beads of a few microns diameter, can be clearly seen by micrographic analysis and may be more carefully established by other techniques. In addition to macropores, the silica beads typically possess a mesoporous structure (in the case of the Merck columns, a total porosity of 80% is claimed, which is made up of macro- and mesopores, the latter of which are on the order of 13 nm in diameter).⁵

### (b) Applications of Monolithic Silicas to Bioaffinity Chromatogrpahy

Bioaffinity chromatography has been used widely for sample purification and cleanup,⁶ chiral separations,⁷ on-line proteolytic digestion of proteins,⁸ development of supported biocatalysts,⁹ and more recently for screening of compound libraries via the frontal affinity chromatography method.^{10,11} In all cases, the predominant method used to prepare protein-loaded columns has been based on covalent or affinity coupling of proteins to silica beads. However, coupling of proteins to beads has several limitations, including; loss of activity upon coupling (due to poor control over protein orientation and conformation), low surface area, potentially high backpressure (which may alter K_{d} values¹²), difficulty in loading of beads into narrow bore columns, difficulty in miniaturizing to very narrow columns (<50 mm i.d.), and poor versatility, particularly when membrane-bound proteins are used.¹¹

In recent years it has been shown that a very mild and biocompatible sol-gel processing method can be used to entrap active proteins within a porous, inorganic silicate matrix.¹³ In this method, a two-step processing method is used wherein a buffered solution containing the protein is added to the hydrolyzed silica sol to initiate gelation under conditions that are protein-compatible.¹⁴ Numerous reports have appeared describing both fundamental aspects of entrapped proteins, such as their conformation,^{15,16,17} dynamics,^{18,19,20} accessibility,²¹ reaction kinetics,²² activity,²³ and stability,²⁴ and their many applications for catalysis and biosensing.^{13,14} A number of reports also exist describing sol-gel based immunoaffinity columns,²⁵ and enzyme-based columns²⁶ although in all cases these were formed by crushing protein-doped silica monoliths and then loading the bioglass into a column as a slurry.

Very recent work on the development of protein-doped monolithic sol-gel columns has appeared from the groups headed by Zusman²⁷ and Toyo'oka.²⁸ Zusman's group have developed columns using glass fibers covered with sol-gel glass as a new support for affinity chromatography. Toyo'oka's group have used capillary electrochromatography (CEC) to both prepare protein-doped sol-gel based columns and to elute compounds. These monoliths were derived solely from TEOS or TMOS using a very high water:silicon ratio, resulting in a loosely packed monolith with large pores to allow flow of eluent. While this is a significant advance, all chromatography was done using electroosmotic flow (CEC), which separates compounds on the basis of a combination of charge, mass and affinity, and is less compatible with MS detection due to the high ionic strength of the eluent. Also, these authors did not examine the interaction of potential inhibitors with entrapped proteins on-column. This is a particularly important issue given the emergence of high throughput screening (HTS) methods based on immobilized enzymes.^{10,11,29}

Improved biocompatibility in monolithic meso- and macroporous silicas arises from the use of polyol derivatives of the silicon-based starting materials rather than TEOS or TMOS. The groups of Brook and Brennan have described the preparation of a series of polyolsilanes based on glycerol,³⁰ sorbitol and related materials.³¹ Free proteins and protein-containing liposomes are dramatically more stable in silicas derived from these species than in analogous silicas prepared from TEOS.³² Porosity in these systems can be induced by the addition of water soluble polymers, such as poly(ethylene oxide).³³

While silica has been widely used for protein entrapment, monolithic silica materials are only stable between pH values of 2.5 and 7.5,³⁴ due to their tendency to dissolve at basic pHs. Erosion of silica is exacerbated by the presence of phosphate-based buffers.³⁵ The gels are also relatively brittle, particularly when formed as a macroporous material.

### (c) Utilization of Titania as Chromatographic Support

The use of sol-gel techniques provides an exceptional degree of morphological control in the preparation of silica. Thus, total porosity, pore size and shape, regularity of pore distribution, etc. can be manipulated using a variety of starting materials, reaction conditions and dopants.³⁶

Titania is also readily formed using sol gel processes.³⁷ A variety of researchers have demonstrated effective synthesis of amorphous titania from standard mono-functional alcohols, usually ethanol, isopropanol or butanol derivatives. Titania based materials have excellent pH stability,³⁸ thermal stability³⁹ and superior mechanical strength compared to silica.⁴⁰ An additional advantage of using titania as a protein entrapment medium is its ability to selectively adsorb organophosphate compounds, such as nucleotides⁴¹ and phospholipids,⁴² allowing it to separate phosphate containing compounds and phosphorylated proteins.⁴³ Furthermore, titania is amphoteric, allowing it to be an anion- and cation exchanger at acidic pH and alkaline pH, respectively, whereas silica can only act as a cation exchanger.⁴⁴

Titania has previously been used to entrap enzymes in a thin film format for biosensor applications.^{45,46} However, although 15 years have passed since the first protein-doped silica monoliths were developed, there is still no report on the development of protein-doped titania monoliths, even though titania possesses many advantages compared to silica, as noted above. In part, this is due to the nature of the common titania precursor, titanium(IV) isopropoxide (Ti(OiPr)₄), the hydrolysis/condensation kinetics of which are very rapid and difficult to control. Another issue is the inherent ability of the isopropyl alcohol byproduct to denature proteins. Finally, for chromatographic applications, synthetic control over porosity is required. Thus, the challenge is to develop a flexible, protein-friendly sol-gel route to make titania-based monoliths with well-defined pore structures.

Alkoxytitanium species exhibit dramatically higher reactivity than alkoxysilanes toward water, which generally leads to the precipitation of the titanium precursor rather than the formation of a polymeric gel. Thus, formation of titania monoliths generally requires chelating ligands such as acetylacetone,⁴⁷ stearic acid,⁴⁸ citric acid,⁴⁹ carboxylic acid,^{50,51,52} alkanolamines,⁵³ ethylene glycol,^{54,55} diols,⁵⁶ or glycerol^{57,58} to attenuate the reactivity of the titanium precursor by stabilizing a high coordination state of titanium.^{37,47,59} Even though such chelating ligands can effectively reduce the reactivity of titanium precursor, only chemical compositions leading to fast hydrolysis but slow condensation rates lead to polymeric gels, otherwise, colloidal sols, gels or precipitates are formed.⁵⁹ As a result, there are only a few reports describing the formation of titania-based sol-gel monoliths.^{60,61,62} However, even in these cases large amounts of alcohol were used to dilute the concentration of titanium precursors, making this sol-gel route unsuitable for protein entrapment.

With respect to the porosity of titania monoliths, Nakanishi has recently reported the formation of macroporous titania starting from colloidal titanium dioxide particles.⁶³ This paper demonstrates that macroporosity can be induced using PEO, as this group has previously demonstrated in a series of elegant papers on silica monoliths.⁶⁴⁻⁶⁷ However, the processes utilized are inherently incompatible with biomolecule incorporation.

### SUMMARY OF THE INVENTION

The present inventors have developed a method of preparing organic polyol-modified titanium precursors useful for the preparation of monolithic biomolecule-compatible titania. The method does not require the use of catalysts and involves the use of organic polyols that are compatible with proteins or other biomolecules. The methods of the invention do not involve the use of compounds, such as Lewis or Brønsted acid catalysts, that may not compatible with proteins.

Accordingly, the present invention includes a method of preparing monolithic biomolecule compatible titania comprising:
(a) combining at least one alkoxytitanium compound with one or more organic polyols under conditions suitable for the reaction of the at least one alkoxytitanium compound with the one or more organic polyols to produce polyol substituted titanium compounds and alcohols, wherein the molar ratio of polyol/alkoxytitanium is greater than about 10; and
(b) hydrolyzing the combination of (a) under conditions suitable for the formation of monolithic titania, wherein said conditions comprise a pH of about 5 to about 9.

In embodiments of the present invention, the organic polyol is biomolecule compatible and is derived from natural sources. In particular, the organic polyol is selected from glycerol, glycols, sugar alcohols, sugar acids, saccharides, oligosaccharides and polysaccharides.

The present invention further relates to novel organic polyol titanium compounds, which are useful as precursors to monolithic biomolecule compatible titania, prepared using the method of the invention. Accordingly, the present invention includes an organic polyol titanium compound prepared by combining one or more alkoxy titanium compounds, one or more organic polyols and, optionally, a solvent, under conditions to form the organic polyol titanium compound, wherein the one or more organic polyols is selected from sugar alcohols, sugar acids, saccharides, oligosaccharides and polysaccharides.

The present invention further includes an organic polyol titanium composition comprising one or more alkyl titanates, one or more organic polyols and, optionally, a solvent, wherein the one or more organic polyols is selected from sugar alcohols, sugar acids, saccharides, oligosaccharides and polysaccharides.

In still further embodiments, the overall pore size, total porosity, morphology and surface area of the titania monoliths can be changed by adding a variety of different additives. In particular, macroporous monolithic titania is obtained when water soluble polymers are used in the methods of the invention. Accordingly, the present invention relates to a method for preparing macroporous monolithic titania comprising:
(a) combining at least one alkoxytitanium compound with one or more organic polyols under conditions suitable for the reaction of the at least one alkoxytitanium compound with the one or more organic polyol to produce polyol substituted titanium compounds and alcohols; and
(b) hydrolyzing the combination of (a) in the presence of one or more water soluble polymers under conditions suitable for phase separation to occur before gelation and for the formation of monolithic titania.

In embodiments of the invention, a macroporous monolithic titania material is obtained by combining a water soluble polymer (for example PEO and derivatives thereof) with polyol-derived titania precursors under conditions where a phase transition, or spinodal decomposition, occurs before the material gels. The phase transition is marked by an increase in turbidity of the precursor/polymer solution.

The mild conditions under which titania materials are prepared using the methods of the present invention are compatible with proteins and other biomolecules. This allows for these types of molecules to be readily incorporated into these siliceous materials for a wide variety of applications. The present inventors have developed biomolecule compatible, macroporous titania materials using the methods of the present invention. It has been shown that these materials can be used for protein entrapment and that capillary columns based on these materials can be prepared that are suitable for pressure driven liquid chromatography and compatible with MS detection. Accordingly, the methods of the present invention further comprise hydrolyzing and condensing the polyol substituted titanium compounds in the presence of one or more biomolecules under conditions suitable for the formation of monolithic titania.

The invention also includes the titania materials prepared using the methods of the invention as well as the use of these materials, for example, but not limited to, in chromatographic applications (particularly with macroporous materials), as bioaffinity supports, biosensors and/or for immobilizing enzymes. Further, the present invention extends to analytical and other types of hardware (for example chromatographic columns, microarrays, bioaffinity columns, etc.) comprising the materials prepared using the methods of the invention.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1 shows the effect of sol components on titania sol gelation behavior (by ratio Ti:Glycerol:H₂O). A: 1 wt % PEO 10 k MW using distilled water. B: 1 wt % PEO 10 k MW in HEPES buffer (pH 7.0, 25 mM). The data were obtained from three measurements on independent samples, and the error bars represent one standard deviation from the mean.

FIG. 2 show the effect of A: buffer concentration (ionic strength). B: pH; on gelation and phase separation times, and C: quantity of 10 k PEO. Not that the x-axes are not to scale. The data were obtained from three measurements on independent samples, and the error bars represent one standard deviation from the mean.

FIG. 3 shows SEM photographs of the morphology of titania monoliths as a function of glycerol/Ti molar ratio, PEO MW and PEO concentration.

FIG. 4 shows thermal analysis of GT16 and GT16-P0.25

FIG. 5 shows Ti/glycerol 1:16 with PEO values ranging from 0-1%. A: BET data. B: Hg-Intrusion porosimetry data.

FIG. 6 shows the kinetics of γ-GT in solution containing PEO, glycerol or isopropanol.

FIG. 7 shows leaching vs. glycerol and water levels for monoliths containing an initial loading of 20 μg of μ-GT per gram of gel. All gels contain 1 wt % of PEO.

FIG. 8 shows the effect of PEO concentration in gel on the amount of γ-GT leaching from a monolith containing a molar ratio of 1:12:16 Ti:glycerol:water as a result of exhaustive washing. Initial protein loading is 20 μg of γ-GT per gram of gel.

FIG. 9 shows the effect of enzyme loading on amount of enzyme retained in a titania gel monolith containing a molar ratio of 1:16:16 Ti:glycerol:water as a result of exhaustive washing. All gels contain 1 wt % of PEO.

FIG. 10 shows γ-GT activity in titania gels as a function of PEO concentration. Gels contain a molar ratio of 1:12:16 Ti:glycerol:water and have an initial loading of 20 μg of γ-GT per gram of gel.

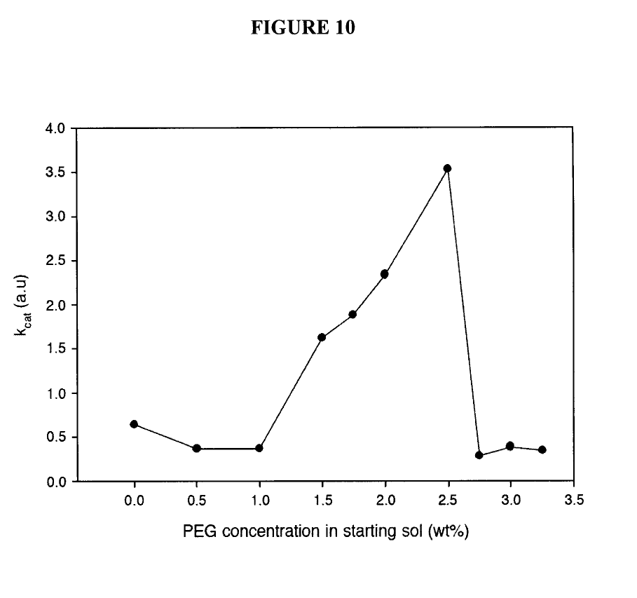

### DETAILED DESCRIPTION OF THE INVENTION

### (I) Definitions

The term “gel” as used herein refers to solutions (sols) that have lost flow.

The term “gel time” as used herein is the time required for flow of the sol-gel to cease after addition of the buffer solution, as judged by repeatedly tilting a test-tube containing the sol until gelation occurred.

The term “cure” as used herein refers to the crosslinking process, the continued evolution of the titania matrix upon aging of the titania following gelation, until the time when the gel is treated (e.g., by washing, freeze drying etc.).

The term “monolithic” as used herein refers to a solid in which the crystal lattice of the entire sample is substantially continuous and unbroken to the edges of the sample.

The term “PEO” as used herein means polyethylene oxide which has the formula HO—(CH₂CH₂O)ₙ—H, wherein n can vary from one to several hundred thousand.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### (II) Polyol-Substituted Titanium Compounds and Monolithic Titania Prepared Therefrom

The present inventors have prepared several different organic polyol-titanium precursors by transesterifying Ti(OiPr)₄ with organic polyols. These precursors are mixtures of materials with well-defined constitutions (i.e., controlled ratios of organic polyols to titanium). Polyols were used to replace isopropoxy groups on titanium to give protein-friendly starting materials and to gain better to control over gelation kinetics which allows for control over the morphology of the resulting titania. These polyols undergo transesterification with in a variety of Ti(OiPr)₄:polyol ratios without the need for catalysts. Accordingly, the present invention includes a method of preparing monolithic biomolecule compatible titania comprising
(a) combining at least one alkoxytitanium compound with one or more organic polyols under conditions suitable for the reaction of the at least one alkoxytitanium compound with the one or more organic polyols to produce polyol substituted titanium compounds and alcohols, wherein the molar ratio of polyol/alkoxytitanium is greater than about 10; and
(b) hydrolyzing the combination of (a) under conditions suitable for the formation of monolithic titania, wherein said conditions comprise a pH of about 5 to about 9.

Alkoxytitanium starting materials that may be used in methods of the invention include those which have the formula: R₄Ti, where R is any alkoxy group that can be cleaved from titanium under the conditions for performing the method of the invention. The R groups need not all be the same, therefore it is possible for one or more of the R groups to be different. In embodiments of the invention the alkyl titanate is a heterogenous or homogenous alkyl titanate derived from ethanol, propanol or butanol. In further embodiments of the invention, all four R groups are selected from methoxy, ethoxy, isopropoxy and n-butoxy. In still further embodiments, the alkyl titanate is tetraisopropoxytitanium.

The organic polyols may be selected from a wide variety of such compounds. By “polyol”, it is meant that the compound has more the one alcohol group. The organic portion of the polyol may have any suitable structure ranging from straight and branched chain alkyl and alkenyl groups, to cyclic and aromatic groups. For the preparation of biomolecule compatible titania, it is preferred for the organic polyol to be biomolecule compatible. By “biomolecule compatible” it is meant that the polyol either stabilizes proteins and/or other biomolecules against denaturation or does not facilitate denaturation. The term “biomolecule” as used herein means any of a wide variety of proteins, peptides, enzymes and other sensitive biopolymers including DNA and RNA, and complex systems including whole plant, animal and microbial cells that may be entrapped in titania. In embodiments of the invention, the biomolecule is a protein, or fragment thereof. In further embodiments of the invention, the biomolecule is in its active form.

It is suitable for the polyol to be derived from natural sources. Particular examples of suitable polyols include, but are not limited to glycerol, glycerol derivatives, glycols, sugar alcohols, sugar acids, saccharides, oligosaccharides and polysaccharides. Simple saccharides are also known as carbohydrates or sugars. Carbohydrates may be defined as polyhydroxy aldehydes or ketones or substances that hydroylze to yield such compounds. The polyol may be a monosaccharide, the simplest of the sugars or carbohydrate. The monosaccharide may be any aldo- or keto-triose, pentose, hexose or heptose, in either the open-chained or cyclic form. Examples of monosaccharides that may be used in the present invention include, but are not limited to, allose, altrose, glucose, mannose, gulose, idose, galactose, talose, ribose, arabinose, xylose, lyxose, threose, erythrose, glyceraldehydes, sorbose, fructose, dextrose, levulose and sorbitol. The polyol may also be a disaccharide, for example, but not limited to, sucrose, maltose, cellobiose and lactose. Polyols also include polysaccharides, for example, but not limited to dextran, (500-50,000 MW), amylose and pectin. Other organic polyols that may be used include, but are not limited to glycerol, propylene glycol and trimethylene glycol. Suitably the polyol is glycerol.

Specific examples of organic polyols that may be used in the methods of the invention, include but are not limited to, glycerol, sorbitol, maltose, trehelose, glucose, sucrose, amylose, pectin, lactose, fructose, dextrose and dextran and the like. In embodiments of the present invention, the organic polyol is selected from glycerol, sorbitol, maltose and dextran. Some representative examples of the resulting polyol modified titanium speces prepared using the method of the invention include diglyceryltitanium (DGT), and tetraglyceryltitanium (DGT). One of skill in the art can readily appreciate that other molecules including simple saccharides, oligosaccharides, and related hydroxylated compounds can also lead to viable titania precursors. Higher molecular weight water soluble polyol polymers do not leach from the titania, once formed, and therefore are a specific embodiment of the invention.

In embodiments of the invention, the conditions suitable for the reaction of the alkoxytitanium compound(s) with the organic polyol(s) to produce polyol substituted titanium compounds and alkoxy alcohols include combining (in any order) the alkoxytitanium compound(s) and organic polyol(s) neat or in the presence of excess polyol or, optionally in the presence of a solvent, and adjusting the temperature so that it is about 0° C. to about 150° C., suitably about 20° C. to about 60° C., more suitably about 25° C. to about 37° C., for about 1 hour to about 72 hours, suitably about 2 hours to about 48 hours. A person skilled in the art would appreciate that reaction times and temperatures may vary depending on the identity and amounts of specific starting materials used and choice of solvent, and could monitor the reaction progress by known means, for example NMR spectroscopy, and adjust the conditions accordingly. The alkoxy alcohol formed as a by-product and/or any solvent used in the method of the invention may optionally be removed by any convenient means, for example, by distillation. The polyol titanium compound product may optionally be isolated by known techniques, for example by evaporation of solvent. In embodiments of the invention, the method of preparing an organic polyol titanium compound further comprises the removal of the alkoxy alcohols. In further embodiments of the invention, the organic polyol titanium compound is used as is, without removal of the alkoxy alcohols or any optional solvent, in the preparation of monolithic titania.

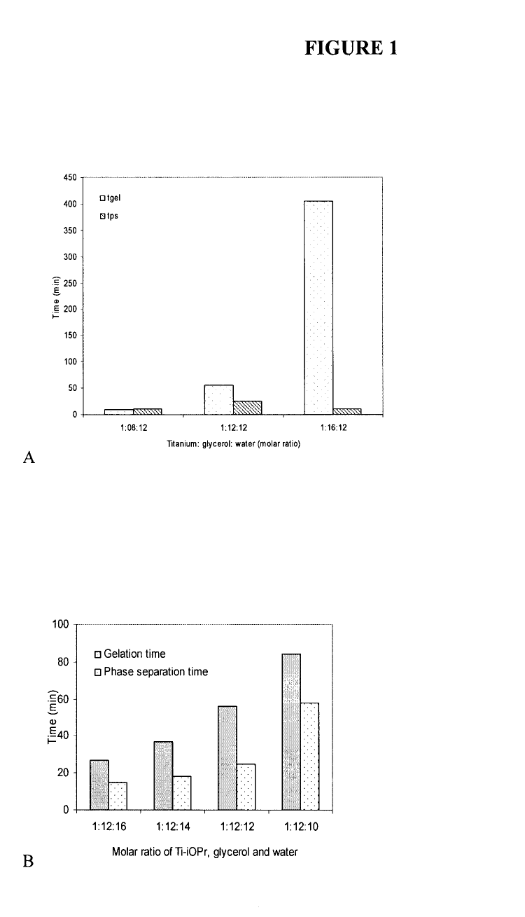

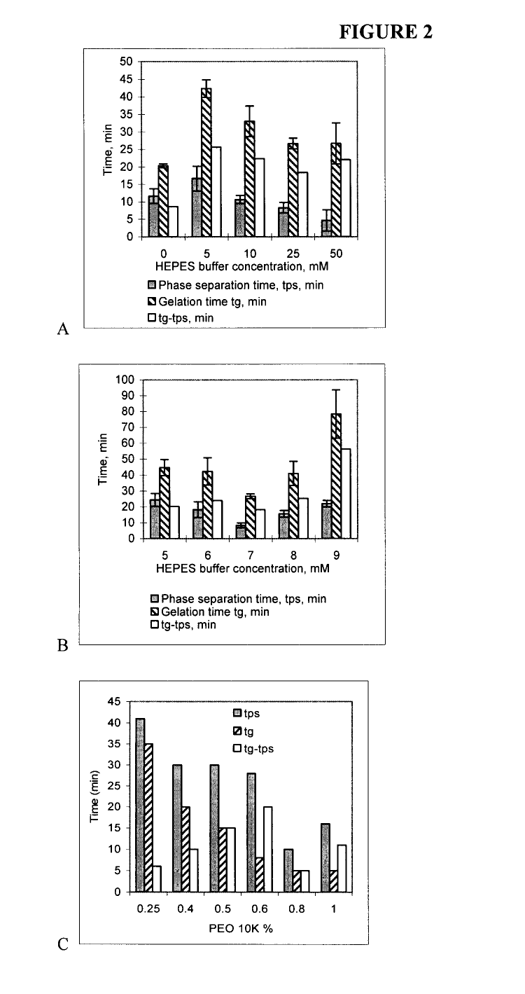

The method of the invention can be carried out in a variety of polyol/titanium ratios. Thus when using one type of polyol, several different polyol titanium species may be formed depending on the starting ratio of polyol to alkoxytitanium compound. The stoichiometric ratio of polyol to titanium in the products affects their rate of hydrolysis and the rate of cure to give titania. Thus, the desirable properties of these compounds include the possibility of tuning the speed with which titania forms, and the ultimate morphology of the titania. Compounds comprising several polyol/titanium ratios were prepared and their hydrolytic behavior examined and described herein (see FIG. 1, FIG. 2, Tables 1-2 and Examples 1-3). It is understood that other polyol titanium species, and ratios of polyols to titanium are readily prepared and not excluded from the scope of the present invention.

In embodiments of the invention, in the absence of additives, monolithic titania may be obtained when the molar ratio of polyol/alkoxytitanium is greater than about 10, suitably greater than about 15 and less than about 35, more suitably about 16. A person skilled in the art would appreciate that the ratio of polyol to alkoxytitanium compound may vary depending on the identity and amounts of specific starting materials, choice of solvents and also on whether or not additives will be used in the subsequent hydrolysis and condensation reaction.

The hydrolysis and condensation of the polyol titania precursors may suitably be carried out in aqueous solution. Suitably, a homogenous solution of precursor, in water is used. Sonication may be used in order to obtain a homogeneous solution. The pH of the aqueous solution of polyol titania precursor may then be adjusted so that formation of a gel (the monolith) occurs. Suitably, the pH may be in the range of about 5-9. The pH may be adjusted by the addition of suitable buffer solutions. For the embedding of biomolecules into the gel, the buffer may further comprise the desired biomolecule.

The invention further includes titania monoliths prepared using the method of the invention. The titania monoliths prepared using the method of the invention are desirably biocompatible as they do not contain any residual catalysts (for example acids or Lewis acidic metal salts) from the preparation of the polyol titanium species. Accordingly, the monoliths may further comprise a biomolecule.

Several factors affect the rate of gelation of polyol modified titania precursors including the ratio of polyol to titanium in the starting materials, the ratio of water to titanium species used in the sol-gel chemistry, the presence of other diluents including alcohols, and the ionic strength of the water. The higher the polyol/titanium stoichiometric ratio in the starting material, the slower is the rate of gelation (e.g., the rate of gelation followed the order: Ti(glycerol)₂<Ti(glycerol)₄<Ti(glycerol)₄+12 equivalents of glycerol (see FIG. 1, Table 1, Table 2, Table 4). Of course, the gelation rates are also dependent on the nature of the container and the exposed surface area (where comparisons were made in the results below, they were made under identical experimental conditions).

The resulting gel may be allowed to cure or age for sufficient period of time. A person skilled in the art can determine this time depending on the desired application for the titania material. The term “cure” or “age” means the continued evolution of the titania matrix upon aging of the titania following gelation. Once the material is sufficiently cured, it may be dried before use. The material may be molded into any desired shape, for example, films, spots, fibres, pellets, granules, tablets, rods and bulk, as the solution becomes viscous but before it becomes completely gelled.

The gelation can also be retarded by the addition of extra polyols to the aqueous media. Performing the hydrolysis of tetraglyceryl titanium under otherwise identical conditions in the presence of additional glycerol clearly showed this effect (see Tables 1, 2, 4 FIGS. 1,20). Thus, it is possible to control the rate of gelation by addition of polyols, water concentration and pH.

The mild conditions under which titania materials are prepared using the methods of the present invention are compatible with proteins and other biomolecules. This allows for these types of molecules to be readily incorporated into these siliceous materials for a wide variety of applications.

In certain embodiments of the invention, there is included an organic polyol modified titanium compound prepared by combining an alkoxytitanium compound with an organic polyol under conditions suitable for the reaction of the alkoxytitanium compound with the organic polyol to produce polyol substituted titanium and alcohols, wherein the organic polyol is selected from a sugar alcohol, sugar acid, saccharide, oligosaccharide and polysaccharide. In particular, the organic polyol modified titanium compound comprises sorbitol, maltose, trehelose, glucose, sucrose, amylose, pectin, lactose, fructose, dextrose or dextran. The present invention further includes a composition comprising an alkoxytitanium compound, an organic polyol and, optionally a solvent, wherein the organic polyol is selected from a sugar alcohol, sugar acid, saccharide, oligosaccharide and polysaccharide. In particular, the organic polyol modified titanium compound comprises sorbitol, maltose, trehelose, glucose, sucrose, amylose, pectin, lactose, fructose, dextrose or dextran.

The present invention provides an example of polyol titanium compounds and compositions which lack acidic or other catalytic contaminants. Such contaminants can affect the titania gelation and cure, and also may not be compatible with biomolecules. Further, the polyol titanium species of the present invention possess characteristics that allow the morphology of the resulting titania to be controlled. Accordingly, in further embodiments of the invention the organic polyol titanium species is free of acidic and other catalytic contaminants. By “free of acidic and other catalytic contaminants” it is meant that the titanium species contains less than 5%, preferably less than 2%, most preferably less than 1%, of acids and other catalytic components. By “acids and other catalytic components” it is meant any such species that is used to catalyze the hydrolysis and condensation of alkoxytitanium compounds and alcohols. Specific examples of such species include Brønsted acids, such as hydrochloric acid, Lewis acids and other catalysts such as poly(antimony(III) ethylene glycoxide.

In specific embodiments of the present invention, there is included an organic polyol titanium species selected from the group consisting of diglyceryltitanium or tetraglyceryltitanium (as found in Tables 1-2).

The present invention further includes an organic polyol titanium composition consisting of one or more alkoxytitanium species, one or more organic polyols and, optionally, a solvent. In suitable embodiments of the invention the organic polyol is biomolecule compatible.

### (III) Preparation of Macroporous Monolithic Titania

The present inventors have developed methods to control the morphology of titania materials derived from organic polyol modified titanium. Specifically, it has been found that the addition of higher molecular weight PEO, or other water soluble polymers, to organic polyol-based sols under conditions where a phase transition, or spinodal decompostion, occurs before gelation, leads to macroporous monolithic titania material. Accordingly, a route to titania materials that are readily formed over a wide range of pHs and which may be prepared at ambient or slightly higher (e.g., 37° C.) temperatures, without the necessity for heat curing or air drying, has been developed. As a result, it is possible to dope these titania materials with a variety of species, in particular biomolecules such as proteins.

Accordingly, the present invention relates to a method for preparing macroporous monolithic titania comprising:
(a) combining at least one alkoxytitanium compound with one or more organic polyols under conditions suitable for the reaction of the at least one alkoxytitanium compound with the one or more organic polyol to produce polyol substituted titanium compounds and alcohols; and
(b) hydrolyzing the combination of (a) in the presence of one or more water soluble polymers under conditions suitable for phase separation to occur before gelation and for the formation of monolithic titania.

The water soluble polymer may be selected from any such compound and includes, but is not limited to: polyethers, for example, polyethylene oxide (PEO), polyethylene glycol (PEG), polypropylene glycol (PPG) or polypropylene oxide (PPO); poly alcohols, for example, poly(vinyl alcohol); polysaccharides; poly(vinyl pyridine); polyacids, for example, poly(acrylic acid); and polyacrylamides e.g. poly(N-isopropylacrylamide) (polyNIPAM). Suitably the one or more water soluble polymers are selected from PEO, PEG, polyNIPAM and PAM. More suitably, the water soluble polymer is PEO. By “water soluble” it is meant that the polymer is capable of being formed into an aqueous solution having a suitable concentration. It should be noted that the terms “oxide” (as in poly(ethylene oxide)) and “glycol” (as in poly(ethylene glycol)) may be used interchangeably and the use of one term over the other is not meant to be limiting in any way.

Alkoxytitanium starting materials that may be used in methods of the invention include those which have the formula: R₄Ti, where R is any alkoxy group that can be cleaved from titanium under the conditions for performing the method of the invention. The R groups need not all be the same, therefore it is possible for one or more of the R groups to be different. In embodiments of the invention the alkyl titanate is a heterogenous or homogenous alkyl titanate derived from ethanol, propanol or butanol. In further embodiments of the invention, all four R groups are selected from methoxy, ethoxy, isopropoxy and n-butoxy. In still further embodiments, the alkyl titanate is tetraisopropoxytitanium.

The organic polyols may be selected from a wide variety of such compounds. By “polyol”, it is meant that the compound has more the one alcohol group. The organic portion of the polyol may have any suitable structure ranging from straight and branched chain alkyl and alkenyl groups, to cyclic and aromatic groups. For the preparation of biomolecule compatible titanias, it is preferred for the organic polyol to be biomolecule compatible. By “biomolecule compatible” it is meant that the polyol either stabilizes proteins and/or other biomolecules against denaturation or does not facilitate denaturation. The term “biomolecule” as used herein means any of a wide variety of proteins, peptides, enzymes and other sensitive biopolymers including DNA and RNA, and complex systems including whole plant, animal and microbial cells that may be entrapped in titania. In embodiments of the invention, the biomolecule is a protein, or fragment thereof.

It is suitable for the polyol to be derived from natural sources. Particular examples of suitable polyols include, but are not limited to glycerol, glycerol derivatives, glycols, sugar alcohols, sugar acids, saccharides, oligosaccharides and polysaccharides. Simple saccharides are also known as carbohydrates or sugars. Carbohydrates may be defined as polyhydroxy aldehydes or ketones or substances that hydroylze to yield such compounds. The polyol may be a monosaccharide, the simplest of the sugars or carbohydrate. The monosaccharide may be any aldo- or keto-triose, pentose, hexose or heptose, in either the open-chained or cyclic form. Examples of monosaccharides that may be used in the present invention include, but are not limited to, allose, altrose, glucose, mannose, gulose, idose, galactose, talose, ribose, arabinose, xylose, lyxose, threose, erythrose, glyceraldehydes, sorbose, fructose, dextrose, levulose and sorbitol. The polyol may also be a disaccharide, for example, but not limited to, sucrose, maltose, cellobiose and lactose. Polyols also include polysaccharides, for example, but not limited to dextran, (500-50,000 MW), amylose and pectin. Other organic polyols that may be used include, but are not limited to glycerol, propylene glycol and trimethylene glycol, suitably glycerol.

Specific examples of organic polyols that may be used in the methods of the invention, include but are not limited to, glycerol, sorbitol, maltose, trehelose, glucose, sucrose, amylose, pectin, lactose, fructose, dextrose and dextran and the like. In embodiments of the present invention, the organic polyol is selected from glycerol, sorbitol, maltose and dextran. Some representative examples of the resulting polyol modified titanium speces prepared using the method of the invention include diglyceryltitanium (DGT), and tetraglyceryltitanium (DGT). One of skill in the art can readily appreciate that other molecules including simple saccharides, oligosaccharides, and related hydroxylated compounds can also lead to viable titania precursors.

The hydrolysis and condensation of the organic polyol derived titanium species in the presence of additives to form monolithic titania typically occurred upon standing of the reagents in aqueous solution or with sonication to assist in dissolution. In suitable embodiments of the invention, the additives are added as solutions in suitable buffers. The aqueous solution may be adjusted to a pH in the range of 5-9 (and may be tailored to the biomolecule, if any is to be entrained in the matrix), using a buffer, for example Tris buffer, to initiate hydrolysis and condensation. In an embodiment of the invention, the pH is adjusted so that it is in a range of about 5-9. The resulting solution will eventually gel (lose the ability to flow) and the material may be allowed to cure or age for sufficient period of time. A person skilled in the art can determine this time depending on the desired application for the titania material. The term “cure” or “age” means the continued evolution of the titania matrix upon aging of the titania following gelation. Once the material is sufficiently cured, it may be dried before use. The material may be molded into any desired shape, for example, films, spots, fibres, monoliths, pellets, granules, tablets, rods and bulk, as the solution becomes viscous but before it becomes completely gelled.

The conditions where a phase transition occurs before gelation may vary depending mainly on the identity of the water-soluble polymer. When the water-soluble polymer is PEO, in order for phase transition to occur before gelation, it is suitable that the non-functionalized PEO be of relatively high molecular weight (MW), for example greater than about 10,000 g/mol, although higher MW materials, greater than about 100,000 g/mol, may also be used and at relatively high concentration, for example from weight percents ranging from about 0.1 wt % up to about 1% for 10,000 g/mol PEO, but low concentrations for higher MW PEO, such as from about 0.001 wt % to about 0.1 wt % for 100,000 g/mol MW PEO. In an embodiment of the invention, the MW of the PEO is between about 10,000 and 100,000 g/mol.

A person skilled in the art can readily determine when a phase transition has occurred, for example, by observing the evolution of turbidity in the sol. As used herein, the time when the solution became totally opaque was recorded as the phase separation time (tₚₛ) and the time with the opaque phase lost its ability to flow was recorded as the gel time (t_{gel}).

Several factors affect the rate of gelation of polyol modified titania precursors including the ratio of polyol to titanium in the starting materials, the ratio of water to titanium species used in the sol-gel chemistry, the presence of other diluents including alcohols, and the ionic strength of the water. The higher the polyol/titanium stoichiometric ratio in the starting material, the slower is the rate of gelation (e.g., the rate of gelation followed the order: Ti(glycerol)₂<Ti(glycerol)₄<Ti(glycerol)₄+12 equivalents of glycerol (see FIG. 1, Table 1, Table 2, Table 4). Of course, the gelation rates are also dependent on the nature of the container and the exposed surface area (where comparisons were made in the results below, they were made under identical experimental conditions). In embodiments of the invention, in the presence of additives, monolithic titania may be obtained when the molar ratio of polyol/alkoxytitanium is greater than about 10, suitably greater than about 15 and less than about 35, more suitably about 12. A person skilled in the art would appreciate that the ratio of polyol to alkoxytitanium compound may vary depending on the identity and amounts of specific starting materials, choice of solvents and also on the identity and amounts of additives used in the hydrolysis and condensation reaction.

The gelation can also be retarded by the addition of extra polyols to the aqueous media. Performing the hydrolysis of tetraglyceryl titanium under otherwise identical conditions in the presence of additional glycerol clearly showed this effect (see Tables 1, 2 and 4 FIGS. 1 and 20). Thus, it is possible to control the rate of gelation by addition of polyols, water concentration and pH.

The mild conditions under which titania materials are prepared using the methods of the present invention are compatible with proteins and other biomolecules. This allows for these types of molecules to be readily incorporated into these siliceous materials for a wide variety of applications. The present inventors have prepared biomolecule compatible, macroporous titania materials using the method of the present invention. It has been shown that these materials can be used for protein entrapment and that capillary columns based on these materials can be prepared that are suitable for pressure driven liquid chromatography and compatible with MS detection. Accordingly, the methods of the present invention further include hydrolyzing and condensing the combination of at least one alkoxytitanium compound with one or more organic polyols in the presence of one or more biomolecules under conditions suitable for the formation of monolithic titania.

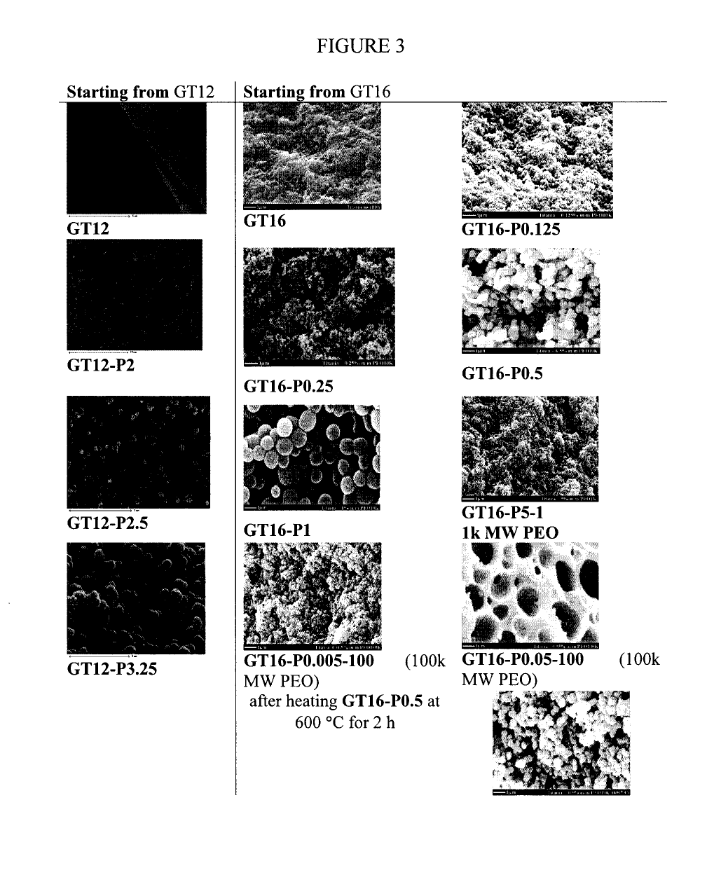

The titania formed as a result of gelation after phase separation consists of small asymmetric beads fused together to create an open structure. The way in which the open structure evolves could be seen by washing unreacted starting material or low molecular weight oligomers from the gel prior to complete reaction of the alkyl titanate. The types of morphologies of available gels can be seen in FIG. 3. The size of the aggregates is a function of the specific recipe used, and in particular depends on the molecular weight and type and weight percent of additive incorporated. With appropriate formulations, the continuous rather than the condensed phase can be made to gel.

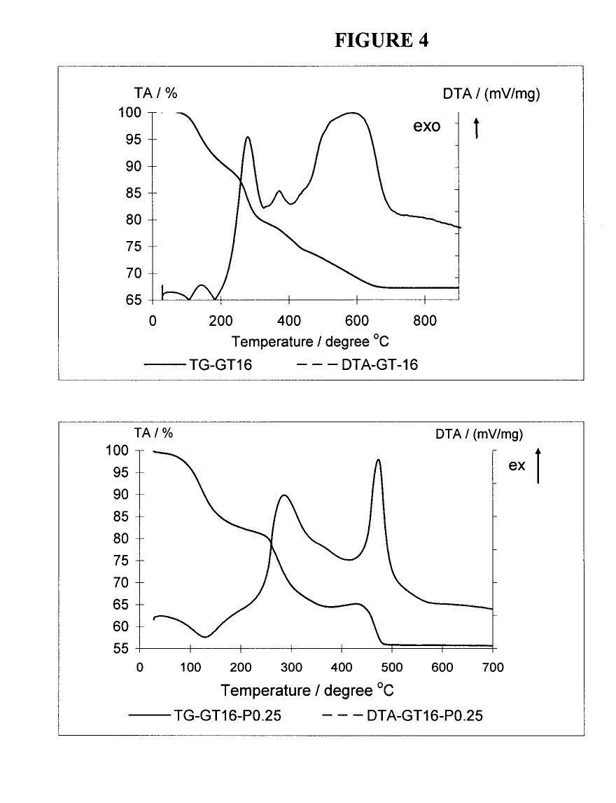

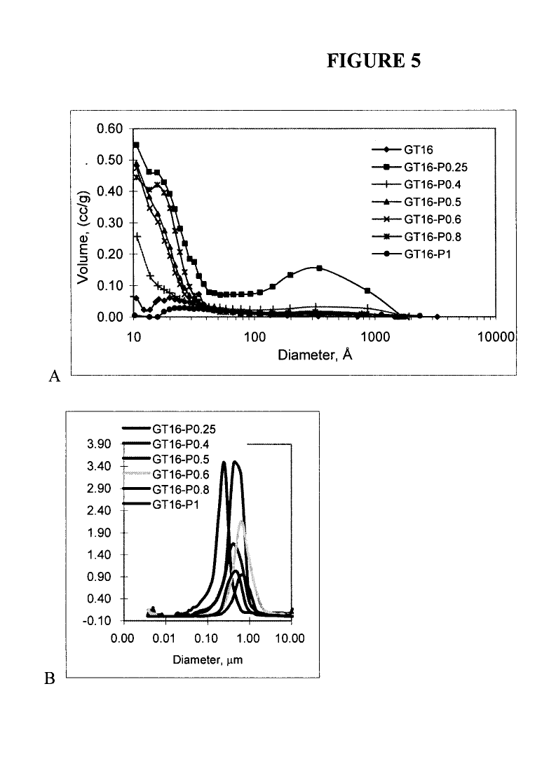

The titania monoliths produced according to the methods of present invention contain residual organic materials used to cause phase separation on cure, as can be seen from FIG. 4. Thermogravimetric analysis (TGA) shows that organic materials are lost as the temperature of the sample is raised above about 100° C. In addition, a conversion from amorphous to crystalline titania occurs at between 250° C. and 600° C. The aggregated titania beads that comprise the monolith are mesoporous in nature. This is clearly seen from the nitrogen absorption data (FIG. 5, Table 3) which shows average pore sizes ranging from about 2.3 nm-7.6 nm for the materials produced after gelation, and about 6.0-12.8 nm after calcination.

The formation of titania by a sol-gel route involves a complex series of hydrolyses and condensations.⁶⁹ When multidentate starting materials are used, such as titanium species derived from glycerol, sorbitol, mannitol, dextrans or other sugar-derived materials, the number of equilibria involved in the reaction cascade from starting materials to titania increases significantly. During this process, low molecular weight materials begin to oligomerize and polymerize. In the absence of significant amounts of other dopants, the final titania monolith forms an optically opaque, white material that contains water, alcohols and other added dopants. The entire process occurs in one phase.

The expedient of adding water soluble polymers, to the original sol complicates the evolution of the titania. While not wishing to be limited by theory, the situation is reminiscent of dispersion polymerization, where after oligomerization, the growing polymer nucleates particle growth.⁷⁰ In this case, the growing titania polymer precipitates from the sol while gelation continues. The specific timing, degree of polymerization, ultimate morphology (including size of the primary particles and aggregates, thickness of the binding titania layers, uniformity of the particle size, pore sizes and porosity) is affected by the quantity, molecular weight and specific molecular characteristics of the additives as shown above.

There are distinctions between the work described here and previous literature reports. These include the nature of the titanium-based starting materials and the interactions of the additives with them. First, the nature of the alkoxy groups on the titanium precursors of the present invention gives these compounds very different pH gelation profiles than titanates derived from simple alkoxy titanium compounds; the residual alcohols of the precursors of the present invention act to plasticize the developing titania network. They also provide an environment which is not destabilizing to entrapped protein. Another distinction is the thermal dependence of the reaction. Gelation occurs at ambient temperature over a wide pH range, again facilitating the incorporation of proteins and other biomolecules in the method of the present invention.

The use of different additives, of different MW and quantities in the sol-gel titania recipe allows the possibility of tuning surface area, total porosity, morphology of the resulting structure, and the magnitude of strength over wide ranges prepared by the sol-gel method from sugar alcohol and related titanates. Another advantage with this combination of reagents over traditional routes is the mild thermal conditions that can be used for its manufacture. In particular, the synthetic route is compatible with the incorporation of proteins and other biomolecules.

The invention also includes the titania materials prepared using the methods of the invention as well as the use of these materials, for example, but not limited to, in chromatographic applications (particularly with macroporous materials), as bioaffinity supports, biosensors and/or for immobilizing enzymes. Further, the present invention extends to analytical and other types of hardware (for example chromatographic columns, microarrays, bioaffinity columns, etc.) comprising the materials prepared using the methods of the invention.

The present invention also extends to the novel macroporous titania monoliths prepared using the method of the invention. The invention therefore relates to a macroporous titania monolith that is compatible with biomolecules and which is prepared at ambient temperature.

### (IV) Uses

It was noted above that a particular advantage of the methods of the present invention is that they are amenable for the preparation of biomolecule-doped titania materials. Accordingly, the present invention further relates to a method of preparing titania materials comprising combining an organic polyol titania precursor, a biomolecule of interest and an additive under conditions suitable for the hydrolysis and condensation of the precursor to a titania material, wherein the additive is one or more water-soluble polymers

The present invention further relates to the titania material comprising a biomolecule or biological substance entrapped therein wherein the titania material is prepared using the methods described hereinabove.

The titania materials prepared using the methods of the invention, in particular those comprising the addition of water soluble polymers and/or biomolecules, are novel accordingly, the present invention further includes all uses of these materials, including, but not limited to, their use in chromatography, biosensors, immobilizing enzymes, affinity supports and the like. In many applications for these materials, a biological substance has been entrapped within its matrixes.

Accordingly, the present invention includes the use of a titania material comprising an active biological substance entrapped therein, as biosensors, immobilized enzymes or as affinity chromatography supports. The present invention also includes a method for the quantitative or qualitative detection of a test substance that reacts with or whose reaction is catalyzed by an active biological substance, wherein said biological substance is encapsulated within a titania material, and wherein said titania material is prepared using a method of the invention. The quantitative/qualitative method comprises (a) preparing the titania material comprising said active biological substance entrapped within a porous, titania matrix prepared using a method of the invention; (b) bringing said biological-substance-containing titania material into contact with a gas or solution, suitably an aqueous solution, comprising the test substance; and (c) quantitatively or qualitatively detecting, observing or measuring the change in one or more characteristics in the biological substance entrapped within the titania material or, alternatively, quantitatively or qualitatively detecting, observing or measuring the change in one or more characteristics in the test substance. Such tests may be performed in various morphologies that will be readily understood by those skilled in the art. Without limitation, these can include microarrays, such as would be achieved using a pinspotter.⁷¹

In particular, the invention includes a method, wherein the change in one or more characteristics of the entrapped biological substance is qualitatively or quantitatively measured by spectroscopy, utilizing one or more techniques selected from the group consisting of UV, IR, visible light, fluorescence, luminescence, absorption, emission, excitation and reflection.

Also included is a method of storing a biologically active biological substance in a titania matrix, wherein the biological substance is an active protein or active protein fragment, wherein the titania matrix prepared using a method of the invention.

The macroporous titania monoliths prepared using the method of the invention are especially useful in chromatographic applications. For the preparation of a chromatographic column, the titania precursor (optionally in hydrolyzed form) and water-soluble polymer (and other additives) may be placed into a chromatographic column before phase transition and gelation occurs.

The present invention therefore relates to a method of preparing a monolithic titania chromatographic column comprising placing a solution comprising an organic polyol titania precursor and one or more additives selected from water-soluble polymers in a column under conditions suitable for a phase transition to occur before gelation.

Some of the additives can be removed or eluted prior to chromatography by rinsing with an appropriate solvent, such as water and/or alcohol. The column may be further prepared by methods such as supercritical drying or the use of a reagent such as a coupling agent to modify the surface of the exposed titania. The monolith may also be stored with the additives interspersed within.

In embodiments of the invention, the titania monolith prepared using the method of the invention is further derivatized to allow tailoring of the monolith for a variety of chromatographic separations. For example, a surface may be incorporated into the monolith that is useful for reverse phase chromatography. Such surfaces may comprise long chain alkyl groups or other non-polar groups. Such derivatization may be done by reacting the Ti—OH or Ti—OR groups on the titania with reagents that convert these functionalities to surface linkages to other organic groups such as alkyls, aryls or functional organic groups (e.g. carboxylates or amines). In still further embodiments, the other organic groups are chiral molecules that facilitate the separation of chiral compounds. These derivatizations are known in the art and are included within the scope of the present invention.

The present invention also includes chromatographic columns comprising the titania monoliths prepared as described herein. Accordingly the invention includes a chromatographic column comprising a titania monolith prepared by combining an organic polyol titanium precursor and one or more water-soluble polymers under conditions where a phase transition occurs before gelation.

In addition, the invention includes the use of a titania monolith prepared using a method of the invention and comprising an active biological substance entrapped therein, as chromatographic columns, biosensors, immobilized enzymes or as affinity chromatography supports. Therefore, the present invention relates to the use of a titania monolith comprising an active biological substance entrapped therein to quantitatively or qualitatively detect a test substance that reacts with or whose reaction is catalyzed by said encapsulated active biological substance, and wherein said titania monolith is prepared using a method of the invention.

Also included is a method for the quantitative or qualitative detection of a test substance that reacts with or whose reaction is catalyzed by an active biological substance, wherein said biological substance is encapsulated within a titania monolith, and wherein said titania monolith is prepared using a method of the invention. The quantitative/qualitative method comprises (a) preparing a titania monolith comprising said active biological substance entrapped within a porous, titania matrix prepared using the method of the invention; (b) bringing said biological-substance-comprising titania monolith into contact with a gas or solution, suitably an aqueous solution, comprising the test substance; and (c) quantitatively or qualitatively detecting, observing or measuring the change in one or more characteristics in the biological substance entrapped within the titania monolith or, alternatively, quantitatively or qualitatively detecting, observing or measuring the change in one or more characteristics in the test substance.

In particular, the invention includes a method, wherein the change in one or more characteristics of the entrapped biological substance is qualitatively or quantitatively measured by spectroscopy, utilizing one or more techniques selected from the group consisting of UV, IR, visible light, fluorescence, luminescence, absorption, emission, excitation and reflection.

### (V) Specific Application to Bioaffinity Chromatography

The present inventors have developed biocompatible, bimodal macroporous titania materials that can be used for biomolecule (e.g. protein) entrapment and have shown that capillary columns based on this material can be prepared that are suitable for pressure driven liquid chromatography and are compatible with mass spectral (MS) detection. The columns were prepared using a mixture of the biomolecule-compatible titania precursor tetraglyceryltitanium (DGT), polyethylene oxide (PEO, MW 10,000), and a buffered solution of the biomolecule of interest to provide bioaffinity sites within the column. The resulting sol mixture was loaded into fused silica capillaries (150-250 μm i.d.), whereupon phase separation of PEO occurred followed by gelation of the titania. The phase separation of the polymer from the titania resulted in a bimodal pore distribution which produced large macropores (>0.1 μm) to allow good flow of eluent with minimal backpressure, and mesopores (ca. 3-5 nm diameter) that retained a significant fraction of the entrapped protein.

Accordingly, the present invention relates to a method of preparing a monolithic macroporous titania column having an active biomolecule entrapped therein comprising combining:

a) a polyol-derived titania precursor;

b) one or more water soluble polymers

c) a biomolecule;

under conditions wherein a phase separation occurs before gelation.

In embodiments of the present invention, the monolithic titania is prepared directly in a chromatographic column. The organic polyol titania precursor may be hydrolyzed, for example by dissolution in aqueous solution with optional sonication, filtered to remove unwanted particulates if necessary, and the hydrolyzed precursor may then be combined with buffered solutions of the one or more additives, biomolecule and any further additives. The resulting mixture may then be transferred to a column before phase separation and gelation occur. In embodiments of the invention, the column is a capillary column.

The present invention further relates to a chromatographic column prepared by combining a polyol-titanium derived titania precursor with one or more additives, a biomolecule under conditions wherein a phase separation occurs before gelation. Also included within the scope of the present invention is the use of this column, for example but not limited to, in methods for immunoaffinity chromatography, sample cleanup, solid phase extraction or preconcentration of analytes, removal of unwanted contaminants (for example by antibody binding), solid phase catalysis and frontal affinity chromatography (with or without mass spectral detection).

The following non-limiting examples are illustrative of the present invention:

### EXAMPLES

### Materials and Methods for Examples 1-3

Chemicals: Titanium isopropoxide, glycerol (99.5%, anhydrous), and poly(ethylene oxide) (PEO) (average molecular weight 1000, 10000 or 100000) were purchased from Aldrich. All water was distilled and deionized using a Milli-Q synthesis A10 water purification system. All other reagents were of analytical grade and were used as received.

### Procedures:

Formation of Titania Monoliths: Meso and macroporous titania materials were formed by a variety of routes. The nomenclature used throughout this report is as follows. Compounds are named by the glycerol/titanium molar ratio, e.g., glycerol:Ti 12:1=GT12. The presence of 10 k MW PEO in the sol is denoted by the presence of a P followed by the weight concentration in the sol. For example, glycerol/Ti/PEO 16:1:0.125=GT16-P0.125. Titania samples prepared with PEO MW other than 10 k are noted explicitly in the Tables.

Titania sols were prepared by mixing titanium (IV) isopropoxide and anhydrous glycerol at a specified molar ratio (1:2-1:32) at room temperature for 2 h. Each mixture was independently dissolved in water or buffer to initiate hydrolysis. The optimized formula in the absence of PEO was a Ti:glycerol ratio of 1:16. In this particular system, glycerol (7.35 g, 80 mmol) was added to titanium isopropoxide (1.42 g, 5 mmol) in the absence of other solvents. The resulting mixture was stirred at room temperature for 2 h to give a milky solution. The sol slowly underwent reaction, but if stored at 4° C. was usable for up to one week.

Formation of titania monoliths from Ti/glycerol sols was done using two different procedures.

### Example 1

In Procedure 1 mesoporous samples were prepared that did not contain PEO. As a representative example, PEO-free titania GT16 was formed as follows. To GT16 (8.79 g, 5 mmol of Ti) was added H₂O (1.44 g, 80 mmol, R value of 16); the mixture was sonicated at room temperature until it became a clear homogeneous solution. The mixture was left at room temperature; a white, opaque and homogeneous gel was obtained in about 1 h. The resulting hydrogel was then aged in a closed container for 2 d and soaked in H₂O (10 mL) for 4 h; this process was repeated 9 times, 4 hours each, with fresh water. The gel was then allowed to dry in air to give a yellow, translucent monolith. Monoliths formed with different R values (4-32) were also prepared.

### Example 2

### Prophetic

Potato starch is suspended in water (5% solids) and cooked at 80° C. for 1 hour. After cooling to room temperature, but prior to gelation of the starch, GT16 is added to the starch at various concentrations. Depending on the specific formulations, gelation will occur within several seconds to minutes. The samples are aged as in example 1 and then washed. The monolith is allowed to dry to give translucent or opaque monoliths.

### Example 3

PEO-containing titania monoliths with bimodal meso/macroporous morphologies were prepared by Procedure 2 as described below. Initially, survey experiments to ascertain the effect of glycerol on titania aging times were undertaken with fixed ratios of H₂O to Ti(OiPr)₄ to PEO 10 k MW of 12:1:1 (R=12), while the glycerol ratio was varied from 8-16. A second set of experiments probed the effect of different amounts of water using GT12-P1 (R=10-16). As an example of a typical macroporous titania material, the preparation of GT16-P0.5 is described. To GT16 (8.79 g, 5 mmol of Ti) was added H₂O (0.440 g, 24.4 mmol, R=5), the mixture was sonicated at room temperature until it turned to a clear homogeneous solution. An aqueous solution of poly(ethylene oxide) (0.25 g in 1.0 mL H₂O) was added. The molar ratio of PEO to Ti was 0.5%, with a total water content of 80 mmol. The mixture was left at room temperature; phase separation was observed within 15 min. After a further 15 min a white opaque gel was obtained. The resulting hydrogel was then aged in a closed container for 2 days. The aged gel was soaked in H₂O (10 mL) for 4 h; this process was repeated 9 times, 4 hours each, with fresh water. The gel was then allowed to dry in air to give an opaque monolith. Analogous processes were used to prepare GT12-P1 derivatives, with the exception that the glycerol concentration in the sol was reduced to Ti/glycerol 1:12.

Role of Poly(ethylene oxide): The role of PEO concentration on monolith structure was established using Procedure 2. Initially, PEO of molecular weight 10K was used; the PEO/Ti molar ratio was varied from 0.125 to 1.0% (GT16-P0.125-GT16-P1). The experiments were repeated using PEO of molecular weight 100K; the molar ratio of PEO/Ti was 0.005%-0.05% (GT16-P0.005-100K-GT16-P0.05-100K) and with PEO of molecular weight 1K at 5% concentration (GT16-P5-1K). Other molar ratios were kept constant.
Effect of pH and Buffer on Gelation Kinetics: The roles of pH and buffer concentration on phase separation time, gelation time and monolith structure were also established using more biologically relevant conditions. To the hydrolyzed titanium/glycerol sol GT16 (8.79 g, 5 mmol of Ti) was added a specified amount of aqueous PEO (10 k MW, final concentration ranging from 0.5 to 3.25 wt %) in pH 7.0 HEPES buffer (0.94 mL) at various concentrations. The mixture was sonicated at 0° C. until it turned to a clear homogeneous solution. A solution of PEO of molecular weight 10K in 25 mM HEPES buffer, pH 7.0 (0.4 g in 0.5 mL buffer) was added. The mixture was left at room temperature to gel. The resulting hydrogel was then aged in a closed container for 2 days. The aged gel was soaked in H₂O (10 mL) for 4 h; the water was replaced 9 times. The gel was then allowed to dry in air to give opaque monolith and characterized by porosimetry methods as described below.

The synthetic process is amenable to the preparation of monoliths of a variety of sizes and geometries. Generally, the monoliths were prepared in 20 mL vials with a diameter of about 2.4 cm. After drying, the sample sizes ranged from about 1.1-2.0 cm in diameter, and 0.45 to 1.1 cm thick. The aged, washed and dried materials were rigid, highly macroporous and, not surprisingly relatively friable. The compressive strength of disks prepared using a typical formula GT16-P0.8 were about 30.2±1.9 mN/m.

Crystallinity as a Function of Thermolysis Conditions: GT16 and GT16-P0.5 were prepared in a similar manner using Procedure 2 with or without PEO added. Separate samples of the resulting gels, after washing with water and drying in air, were heated at 200, 480 or 600° C. for 2 h (at the heating rate of 20° C./h), respectively. The resulting materials were characterized by powder X-ray diffraction using a Bruker D8 Advance diffractometer with CuKα₁ radiation to assess the effect of temperature on crystallinity.
Characterization of Titania Materials: Thermogravimetric analysis (TGA) was performed using a Thermowaage STA409. The analysis was performed under air, with a flow rate of 50 mL/min. The heating rate was 10° C./min from room temperature to 900° C. SEM photographs and were observed on JEOL 840 Scanning Electron Microscopy and JEOL 1200EX Transmission Electron Microscopy, respectively.

Prior to N₂ sorption and mercury intrusion porosimetry measurements, all the samples were degassed at 100° C. under vacuum overnight. Nitrogen adsorption-desorption isotherms were recorded on a Quantachrome Nova 2000. The specific surface area was calculated using the multipoint Brunauer-Emmett-Teller (BET) method. Pore size distributions were calculated by the Barrett, Joyner and Halenda (BJH) method. Pore volumes were determined from the amount of N₂ adsorbed at P/P₀=0.99. Macropore intrusion volumes and macropore size distributions were measured by mercury intrusion porosimetry on a Poremaster GT 60 over a pressure range of 0.10-60,000 psia and analyzed using the Washburn Equation.

### Results for Examples 1-3

Titania is similarly formed by a sol-gel process initiated by the hydrolysis of alkyl titanates, of which Ti(OiPr)₄ is exemplary.³⁷ Initially attempted was the preparation of a series of titanium glycerol derivatives (Ti(glycerol)_{y}, y=1-4) using the protocol that was effective with silanes. Transesterification of Ti(OiPr)₄ with glycerol or other sugars was attempted under a wide variety of conditions with, or without, the utilization of solvents such as THF or DMSO. In contrast to the silanes, attempts to transesterify the more Lewis acidic Ti(OiPr)₄ with glycerol led to a milky suspension that was not soluble in water. Thus, it was necessary to form titania directly from a dispersion of Ti(OiPr)₄ in glycerol without removal of the isopropanol. As noted below, the ratio of Ti:glycerol was important in controlling the subsequent condensation kinetics, which in turn were associated with morphological control of the resulting monolith.

Monolithic titania was prepared by the hydrolysis of glycerol —Ti(OiPr)₄ mixtures using two distinct protocols: simple hydrolysis and hydrolysis in the presence of high molecular weight poly(ethylene oxide) (PEO). Extensive experimentation was needed to optimize titania formation from glyceroxytitanium species, and past experience with silicon provided no helpful guidance: for example, condensation/gelation of Si(glycerol)₄ was inconveniently slow, whereas the analogous processes of Ti:glycerol 1:4 were uncontrollably fast.

Gelation time, t_{g}, was defined as the time elapsed between the point when all chemicals were added together and the time when the monolith lost the ability to flow. As the molar ratio of glycerol increased from Ti:glycerol 1:2 to 1:16 at a fixed water concentration, there was an increase in t_{g}. Lower glycerol levels led to exceptionally fast gelation and produced only TiO₂ particulates. Recipes using a higher glycerol molar ratio of 1:32 exhibited retarded condensation rates, but did not exhibit other obvious advantages. The 1:8 ratio mixture gelled over a convenient period of time, but led to a fragile monolith susceptible to cracking. The optimal stoichiometry for an effective and practical titania precursor was found to be about Ti: glycerol; 1:16, GT16, although when PEO was also present in the sol (see below) it was possible to utilize less glycerol (e.g., a 1:12 ratio was suitable; i.e., GT12-P1). The presence of buffer rather than distilled water also affected gelation times (see below). Monoliths prepared at higher glycerol concentrations were more resilient.

The kinetics of monolith formation was examined by hydrolyzing GT16 as the titania precursor at room temperature with varying ratios of water:Ti. The amount of water present was a key factor in controlling the gelation time and resulting morphology of the monolith (Table 1). When the water concentration was kept very low (H₂O:Ti, 4:1), gelation took about 1.5 days. By contrast, increasing the amount of water by a factor of 4 (H₂O:Ti, 16:1) decreased the gelation time by a factor of 35, to one hour.

### Gelation and Phase Separation Behavior in PEO-Doped Gels

The second process for forming titania exploits Nakanishi's utilization of a water soluble polymer, such as poly(ethylene oxide) (PEO), to change the condensation behavior of silica in sol gel processes.^{72, 73, 74} Overlaid on the conversion of sol to gel via condensation is a phase separation process leading to a titanium rich phase that ultimately forms a gel, and a titanium poor phase which is removed at the end of gelation. Nakanishi reports that large molecular weight PEO (>10,000 Da) is necessary to induce macroposity in silica,^{62,64,67} and used 100,000-1,000,000 MW PEO for the formation of macroporous titania.⁶³ Reported below is the formation of monoliths using either 10,000 or 100,000 MW PEO at various concentrations, which were incorporated at different weight ratios into the sol.

Initial experiments reprised the examination of the titanium isopropoxide:glycerol molar ratio on the gelation time, with PEO concentration fixed at 1 wt %, as shown in FIG. 1. In addition to the key gelation time parameter t_{g} (defined above) is the phase separation time, tₚₛ, which is defined as the time required for the transparent sol to become translucent, again relative to the point where all components were mixed (t=0). As shown in FIG. 1A, increasing the titanium:glycerol molar ratio in the starting sol from 1:8 to 1:16 increased the t_{g} from 9 min to 405 min, whereas tₚₛ did not change drastically (ranging from 10 to 25 min). Thus, the difference in gelation and phase separation times, the coarsening time t_{g}−tₚₛ, varies from −1 min at lower glycerol levels (i.e., gelation occurs just prior to phase separation) to as long as 380 min at high glycerol levels. Gelation occurred almost immediately when the titanium:glycerol molar ratio was lower than 1:8, thus ratios lower than this were not investigated when PEO was present. Higher concentrations of glycerol tend to suppress condensation, increasing both t_{g} and tₚₛ. FIG. 1B and Table 2 show the effect of water concentration on the gelation behavior of the titanium sol in the presence of PEO (GT12-P1). Both t_{g} and tₚₛ decreased with increasing water concentration in the titanium sol, consistent with more rapid hydrolysis in the presence of higher levels of water coupled with higher rates of condensation as the glycerol concentration was diluted.

Other Parameters that Affect the Condensation/Aggregation/Gelation Profile

In addition to glycerol and water concentration, parameters such as the PEO concentration, operating temperature, ionic strength and pH were all found to affect the gelation behavior. For example, increasing temperature resulted in faster condensation, similar to what is observed for silica systems. For convenience, ambient temperature was routinely used for the formation of titania. The concentration and molecular weight of PEO was also a strong contributor to both t_{g} and tₚₛ. Both t_{g} and tₚₛ dropped as the concentration of PEO (10 k MW) was increased: there was an increase and then a decrease in t_{g}−tₚₛ (FIG. 2C). The absolute concentration of ethylene oxide monomer units was less important in mediating these changes than the molecular weight. Thus, decreasing the [EO] by a factor of 100—using 0.5% MW 10 k PEO vs. 0.005 MW 100 k PEO—led only to increases of t_{g} and tₚₛ by a factor of 2 (GT16-P0.5 vs. GT16-P0.005-100, Table 2). By contrast, an 8-fold reduction in [EO] using PEO of the same MW (10 k) led to a reduction of 6-fold in the tₚₛ and 3-fold in the t_{g} value, respectively (GT16-P1 vs. GT16-P0.125, Table 2).

Unlike silica, increases in ionic strength tended not to result in significant changes in the gelation times of titania. As the buffer (HEPES) concentration was increased from 5-50 mM, there was a decrease in the t_{g} from 40 to about 30 min. (FIG. 2) ce t_{g}−tₚₛ was higher at all buffer concentrations than when sols were prepared from deionized water (FIG. 2A). The nominal fused particle size was only marginally affected by buffer concentration. As the concentration varied from 5-50 mM, fused particles sizes ranged from about 500-600 nm with no obvious trend being evident). Shifts in pH to more basic conditions led first to a decrease in t_{g} as pH approached neutrality, and then significantly increases in t_{g} at pH 9 (FIG. 2B). The value of tₚ, followed a similar pattern, but to a much lower degree. The difference t_{g}-tₚ, increased with increasing pH. Interestingly, the nominal fused particle size is consistent over the pH range 5-7 (550 nm), and then decreases with increasing pH (pH 8: 400 nm, pH 9: 300 nm).

Monoliths were prepared using this process in a variety of sizes and geometries, including capillary columns, and disks of dimensions as large as 1-2 cm diameter and 0.5-1 cm thick: thin films would typically crack during the shrinkage that accompanied aging and drying.

### Morphology Control in Titania Monoliths

The morphology of the titania gel was affected by presence of 10 or 100 kDa PEO in the sol: it was not possible to form macroporous titania gels starting with 1 k MW PEO (Table 2). With 1:12 Ti/glycerol (GT12) as the starting sol, SEM images of the titania gel in the absence of PEO demonstrated no features (pores or particles) at the resolution of the SEM instrument (FIG. 3). Increasing PEO concentration to 1 wt %, GT12-P1, led to a gel with a rough surface, where mesopores may exist, but there were no detectable macropores observable under SEM. Although the gel monolith in the wet state halls an opaque appearance, suggesting the presence of macropores, the monoliths become translucent after drying, indicating the disappearance of macropores due to either collapse or shrinkage (see below). Increasing the PEO concentration led to a rougher surface, with some detectable macropores that were about a few hundred nanometers in diameter as shown in FIG. 3. When a PEO concentration of 2.25 wt % was utilized, a clearly interconnected gel network was obtained, with throughpores of 1-2 μM in diameter, GT12-P2.25. Titania gel monoliths with interconnected gel networks appeared over a limited range of PEO concentrations, where spinodal decomposition occurs. Further increases of PEO concentration led to a morphology of particle aggregates, with feature sizes (particle size) increasing from about 400 nm to about 800 nm when PEO concentration increased from 2.5 to 3.25 wt %, GT12-P2.5 and GT12-P3.25.

Similar changes were observed with the GT16 series of compounds, but at much lower PEO concentrations (FIG. 3). With this system, macroporous structures were already formed with PEO 10 k MW at concentrations of 0.5%, GT16-P0.5. Comparable morphologies were observed with 100 k MW PEO at even lower concentrations (e.g., GT16-P0.125 (0.125% 10 k PEO)≈GT16-P0.005-100 (0.005% 100 k MW PEO). Nominal fused particle size also increased with PEO content in this series (e.g., GT16-P1: 1150 nm vs. GT16-P0.5: 470 nm, FIG. 3, Table 2). Note that if higher concentrations of high MW (100,000) PEO are utilized, it is possible to gel the continuous (GT16-P0.05-100)) rather than the dispersed phase (GT16-P1).

### Porosimetry Studies

Titania gels were also characterized by nitrogen adsorption-desorption (BET) porosimetry and mercury intrusion data (FIG. 5). Isotherms of all titania gel monoliths were of type IV with a H3 hysteresis loop. Analysis of the nitrogen desorption data using the BJH model (FIG. 5A) showed that the titania gels contained mesopores at all PEO concentrations, with mesopore diameters centered at 2.2 nm. Titania gels derived from Ti/glycerol 1:12 sols containing 2 or 2.25 wt % of 10 k PEO, and gels derived from Ti/glycerol 1:16 sols containing 0.25 wt % of 10 k PEO possessed larger mesopores centered at 33 nm. Pore volume increased with increasing PEO concentration, reaching a maximum pore volume at concentration of 2.25 wt % of 10 k MW PEO for 1:12 Ti/glycerol and 0.25 wt % for 1:16 Ti/glycerol (Table 3). Further increases in PEO concentration lead to a decrease of pore volume, and the disappearance of large mesopores.

Titania gels derived from Ti/glycerol 1:12 containing PEO concentrations higher than 1.75 wt %, and Ti/glycerol 1:16 with PEO concentrations greater than 0.125% appeared to be opaque upon drying at 150° C. overnight, indicating the presence of macropores. These macropores were beyond the detection limit of nitrogen sorption analysis. Therefore, those gels were examined by Hg intrusion porosimetry, with pore size distribution data shown in FIG. 5B and Table 3. In all cases, the titania gels possessed a narrow pore size distribution. Irrespective of the initial sol formulation, the macropore size first increased and then decreased with PEO concentration. A similar trend of increasing followed by decreasing pore size with increases in PEO concentration has been reported for silica systems,³¹ and reflects a change from bicontinuous to particle aggregate morphologies, as demonstrated by the SEM images shown in FIG. 3.

The effects of pH, buffer concentration and PEO concentration on pore size and surface area were examined. As the pH was modified from 5-9, there was initially little effect on porosity, median pore size and total intruded volume until neutrality was approached, at which point all factors began to decrease (see FIG. 2). A similar profile was observed for changes in median pore size as a function of buffer concentration. Pore size dropped as buffer concentration was increased. By contrast, the total surface area increased as a function of buffer concentration. There was no significant change in total intruded volume as a function of buffer concentration.

Highly mesoporous gels were self-supporting monoliths that were somewhat fragile. Thus, rigid disks (after aging and drying) derived from GT16-P0.8 of about 1 cm diameter and 0.5 cm thickness showed compressive strengths of 0.208±0.013 MPa.

### Crystalline Character of Monolithic Titania

The aged materials contained measurable quantities of PEO. Extensive washing was required to remove unbound glycerol and PEO, which otherwise remains sequestered within the monolith. Allowing the samples to dry at 25° C. for 60 days was accompanied by shrinkage of the titania gel, which was more severe than the analogous silica compounds. Shrinkage at room temperature was inversely proportional to the amount of PEO present in the gel (Table 2). The residual PEO and glycerol act to plasticize the gel. However, shrinkage could be induced thermally, even in PEO-rich gels, and is directly correlated with thermal treatments (both time and temperature of exposure) that remove the organic constituents (Table 3), as is clearly seen in the before and after micrographs of a gel calcined at 600° C. (FIG. 3).

Thermolysis of the organically modified titania gel also affects the crystallinity of the matrix. The X-ray diffraction patterns of samples GT16 and GT16-P0.125, prepared under ambient conditions, suggest that these samples are amorphous: no crystalline domains were present. Heating the samples at a relatively low temperature (200° C.) did not change the amorphous structure. However, heating the samples to higher temperatures caused them to convert to a phase of imperfect crystallinity that was dominated by the anatase phase. The XRD patterns of both samples GT16 and GT16-P0.5 after heating at 480° C. for 2 hours showed broad and overlapping peaks at 2θ=38 and 62°. Sample GT16-P0.5, prepared with PEO, showed a higher degree of crystallinity than sample GT16, prepared without PEO. When treated for 2 hours at 600° C., yet higher degrees of crystallinity, consistent with anatase,⁷⁵ were observed. This suggests that the plasticization effects of PEO may facilitate anatase crystallization.

The TEM and selected area electron diffraction (SAD) of samples GT16 and GT16-P0.5 prepared without calcination similarly show the absence of crystalline diffraction rings, whereas after heating at 600° C. for 2 hours both show the presence of an anatase diffraction ring (Table 3). TEM also shows that the crystal size is dependent upon the temperature treatment to which the monolith is exposed and whether PEO was in the sol: for compounds cured at 400° C. for 2 hours, crystal sizes in sample GT16 are slightly bigger than those in sample GT16-P0.5.

DTA data (FIG. 4) of sample GT16 at 594° C. shows a broad exothermic peak (from 468-685° C.), while sample GT16-P0.5 shows a narrow exothermic peak at 464° C. (onset 441° C., end 482° C.). These results suggest that upon heating, the PEO-induced macroporous structure has a higher crystallization rate than the titania prepared without PEO.

The TGA results of samples GT16 and GT16-P0.5 are shown in FIG. 4. For sample GT16, prepared without PEO, there are 3 major zones of mass loss, with a total mass loss of 32.9%. The mass loss below 200° C. is ascribed to desorption of absorbed water (8.8%). The mass loss from 200-350° C. (12.0%), associated with an exothermic peak at 276.6° C., is attributed to the decomposition of absorbed glycerol. The mass loss from 350-600° C. arises from the decomposition of unhydrolyzed organics and the dehydration of titanium oxyhydrate (12.2%).⁷⁶ There are two exothermic peaks at 370 and 594° C. The former peak is attributed to the combustion of unhydrolyzed organics, while the latter is attributed to the crystallization from amorphous to anatase phase.

For sample GT16-P0.5, which was prepared with PEO, mass losses up to 400° C., but particularly associated with a broad exothermic peak at around 270° C., were attributed to the removal of absorbed water, residual organics and PEO (35%). Mass losses from 400-500° C. are consistent with dehydration of titanium oxyhydrate (9%). The exothermic peak at 464.6° C. is attributed to crystallization during conversion of the amorphous to the anatase phase.⁷⁷

### Discussion for Examples 1-3

Formation of a titania gel from Ti(OiPr)₄ and glycerol involves several concurrent events. At a molecular level, hydrolysis and condensation occur to build up oligomeric structures along the pathway Ti—OiPr→TiOH→Ti—O—Ti. When oligomers achieve a certain size, they precipitate from the solution forming primary particles that simultaneously grow and, in the absence of effective particle stabilizers, are captured by larger particles. Ultimately, a monolith is formed when the concentration of grown particles is sufficiently high, and the ability to stabilize large particles is sufficiently low that they flocculate and are subsequently bound together by further condensation/growth processes to form self-supporting spanning clusters. Most of the monoliths prepared have a fused bead structure (FIG. 3), which is characterized both by the nominal size of the particles and by the degree to which the fused particles are monodisperse. The density of packing (macroporosity) and internal void volume (mesoporosity) are also characteristics of the gel. The role of glycerol, PEO and reaction conditions (e.g., pH, buffer concentration) in controlling these effects are examined in turn below.

Alkoxytitanium species undergo hydrolysis and condensation reactions extremely rapidly, much more rapidly than alkoxysilanes. Nakanishi and coworkers seeded their titania sol, also containing Ti(OiPr)₄, with ˜7 nm TiO₂ particles because otherwise gelation and phase separation were too fast to permit controlled monolith formation.⁶³ The presence of polyols, such as glycerol, and use of low water concentrations in mineral sols affects the hydrolysis and condensation kinetics. Overall gelation rates for silica, which are a composite of the rates of hydrolysis, condensation and particle aggregation, are retarded as a function of glycerol concentration.⁷³ Glycerol similarly retards these processes in the formation of TiO₂ monoliths.

Glycerol is used in the titanium-glycerol sol-gel route as a chelating ligand, which decreases the hydrolysis/condensation rate by transesterifying onto the primary titanium precursor (i.e., Ti(OiPr)₄+HOCH₂CHOHCH₂OH→HOCH₂CHOHCH₂OTi(OiPr)₃), forming a less water-sensitive secondary precursor, as proposed by Sanchez and co-workers.57 There is an additional role played by glycerol. Unlike monofunctional alcohols, with which hydrolysis in aqueous solvents is essentially irreversible, the polyol can participate in intramolecular reactions that reform alkyl titanates (Scheme 1). Thus, the presence of polyols additionally distorts the equilibrium for hydrolysis and condensation towards starting materials. Finally, glycerol increases the viscosity in the medium, moderating collisions between growing titania oligomers and particles.

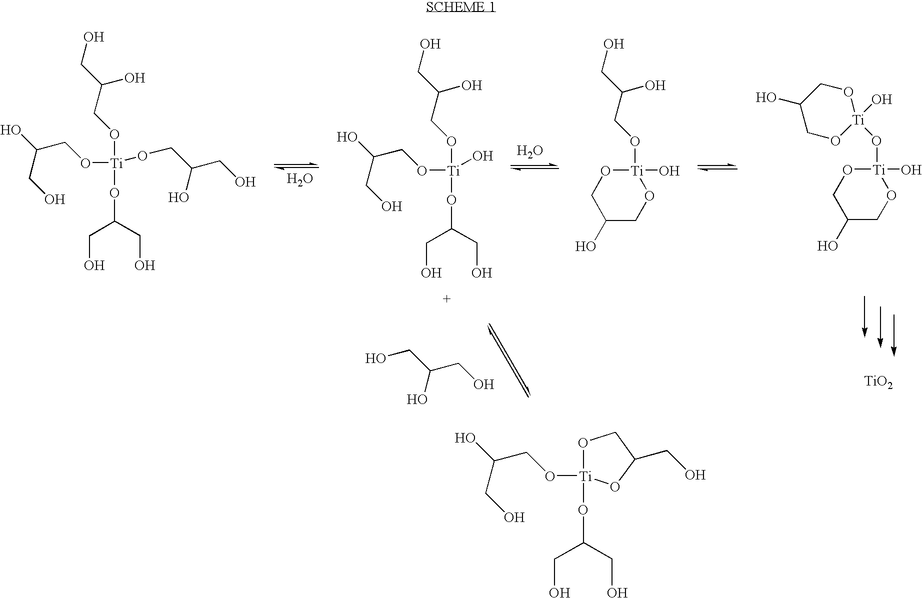

In a silica-based sol-gel system, macroporous morphology can be conveniently obtained in a controlled manner by inducing phase separation in parallel to the sol-gel transition.⁶⁵ The mechanism of phase-separation induced by the addition of PEO in tetramethoxysilane (TMOS) or tetraethoxysilane (TEOS) systems has been extensively discussed.^{65,66} Few studies have previously examined the possibility of controlling titania morphology using this approach. Kajihara et al. have prepared macroporous TiO₂ films using a sol-gel dip-coating method from a titanium alkoxide-based solution containing poly(ethylene oxide).⁶⁷ As noted above, starting from 7 nm TiO₂ particles, Nakanishi formed macroporous titania monoliths by hydrolyzing Ti(OiPr)₄ at very low pHs in the presence of high MW PEO (100,000-1,000,000 MW).⁶³

At higher levels of PEO fewer (larger) particles are formed, which ultimately fuse into a gel. Similarly, higher molecular weight and more viscous PEO leads to more effective flocculation of small particles into larger aggregates that ultimately fuse. By contrast, as the PEO flocculant concentration is reduced, or the PEO MW lowered, more primary particles can independently grow before fusing into aggregates. Glycerol, another viscous, hydrogen bonding material, can amplify this effect, thus similar monoliths can be prepared when some high MW PEO is replaced with glycerol (e.g., GT16-P1).

The kinetics of hydrolysis and gelation, and effects of particle aggregation caused by pH and buffer concentration are subordinate to the flocculating role of PEO. As the buffer concentration increases from 5-50 mM, there is little change in nominal fused particle size, nor in t_{g} or tₚₛ. pH similarly has little effect except at higher pHs (8-9), where more, smaller particles are formed and remain able to grow independently prior to fusion. Note that the pH constraints of traditional methods are completely subverted by the utilization of glycerol. There is no need to use excessively acidic pHs to moderate titania growth. Instead, biocompatible pHs can be utilized. The driving force for both gelation and phase separation is condensation of titanium aggregates. Although the relationship between PEO MW and concentration has been previously noted for silica and titania, the effect has typically been correlated with coarsening time.^{63,78}

Coarsening time t_{g}−tₚ, was not an effective predictor of structure for these monoliths (FIG. 2). For example, comparable low values of t_{g}−tₚ, were observed with formulas containing 0.125% and 0.5% 10 k MW PEO. However, the resulting monoliths are strikingly different, with nominal fused particle sizes of 60 and 475 nm, respectively. More instructive is the t_{g} value. As t_{g} drops, there is a correlation to larger nominal particle sizes, resulting from larger but fewer particles (FIG. 2, FIG. 3). Any constituent in the sol that impedes colloidal particle aggregation leads to more and smaller fused particles and a less macroporous structure. Thus, high pH is associated with increased surface charge and electrostatic stabilization of smaller particles, which are eventually captured at long t_{g} into a tightly fused mass. By contrast, high molecular weight PEO facilitates small particle flocculation, leading to very large particles at short t_{g}. The intersection of molecular and colloidal processes is dependent on the presence of glycerol and water.⁷⁹ Retarding the rate of condensation using glycerol and metering the water content in the sol permits colloidal aggregation processes to compete with titania growth. In the absence of glycerol, it is necessary to provide seed particles whose further reaction and agglomeration leads to macroporous monoliths.⁶³

Increases in the PEO concentration were associated with reduced mesopore and increased macropore size (Table 3). The fusing of large, improperly packed particles engenders the formation of large macropores. Smaller particles pack more efficiently with consequently smaller macropore sizes. At low PEO concentration, the primary particles grow to larger sizes before associating. The nascent mesopores—interstices between particles—will be smaller when the aggregated particles are smaller.

Titania gels formed by sol-gel processes undergo extensive shrinkage. In the absence of PEO, shrinkage of 95% was noted for GT16. The presence of small amounts of PEO moderates shrinkage, but the effect is lost as PEO concentration is increased (Table 2). The plasticization provided by the polymer is more efficacious when particles are small. Monoliths comprised of larger particles, with less surface contact area, were typically more fragile.

A similar role by PEO is played during extensive thermal heating. Gels formed at ambient temperature were amorphous. Upon heating, the onset of crystallization occurs at lower temperature in the sample containing PEO than in a monolith derived from glycerol alone (FIG. 4). At this temperature, 441° C., either residual PEO in the monolith, or a more flexible structure resulting from the presence of PEO during monolith fabrication can facilitate reorganization of the titania into crystalline domains.

### Example 4

Chemicals: The enzyme γ-glutamyl transpeptidase (E.C. 2.3.2.2, lyophilized powder from bovine kidney) was obtained from Sigma (Oakville, ON). Titanium(IV) isopropoxide, L-glutamic acid gamma-(p-nitroanilide) (GPN) and glycylglycine were obtained from Sigma (Oakville, ON). Anhydrous glycerol was purchased from Fluka (Switzerland). Poly(ethylene) glycol (PEO, MW 10 kDa) was obtained from Aldrich (St. Louis, Mo.). Cy5-maleimide mono-reactive dye was purchased from Amersham Biosciences UK limited (Buckinghamshire, England). All water was distilled and deionized using a Milli-Q synthesis A10 water purification system. All other reagents were of analytical grade and were used as received.

### Procedures

Entrapment of γ-GT in Titania Monoliths: Solutions of γ-GT (1 mg·mL⁻¹) were prepared in HEPES.NaOH buffer (pH 7.0, 25 mM). Entrapment of the enzyme in monolithic titania started by adding 1 mmol of titanium isopropoxide (0.30 mL) to anhydrous glycerol at a specified molar ratio (titanium:glycerol=1:8 to 1:16), mixing at room temperature for 2 h, followed by the addition of a specified amount of water to initiate the hydrolysis. To this hydrolyzed sol, aqueous PEO (10 KDa, 6.25 g PEO/g H₂O) and γ-GT (1 mg·mL⁻¹) in pH 7.0, 25 mM HEPES buffer, was added successively with gentle mixing. The final concentration of γ-GT in the sol was 20 μg/g gel, whereas final PEO concentration varied from 0.5 to 3.25 wt %. The mixture containing the enzyme and PEO was subsequently dispensed into microtiter wells (total volume 100 μL, enzyme loading 1.35 μg/well) and allowed to gel (typically 10-50 min, depending on the sol composition). The plate was aged in the dark at 4° C. prior to performing enzyme assays.
Leaching of Entrapped γ-GT: Prior to assaying enzyme activity within monolithic titania, sol-gel entrapped enzyme samples were washed 5 times with buffer solution to remove excess glycerol. The supernatant from each wash was collected to evaluate the immobilization efficiency, which was defined as the ratio of enzyme in the supernatant relative to the amount of enzyme initially added to the sol. Leaching was evaluated by measuring the fluorescence intensity of Cy5-labeled γ-GT in the washing solution, as described by Besanger et al.⁸⁰ Labeling of γ-GT was accomplished by mixing 1 mL of 1 mg·mL⁻¹ γ-GT with 1 mg of Cy5-maleimide dissolved in 50 μL dimethylformamide and allowing the reaction to proceed for 30 min at room temperature and then a further 18 h at 4° C. Unreacted dye was separated from the labeled enzyme by passing the mixture though a Sephadex G25® column. The Cy5 labeled γ-GT was then entrapped in titania monoliths as described above. The fluorescence emission intensity of Cy5 was monitored using a TECAN Safire microwell plate reader with excitation and emission wavelengths of 649 and 670 nm, respectively. The emission intensity from the supernatant was summed and normalized to the emission intensity obtained from a concentration of enzyme equivalent to that which would be obtained for 100% leaching.
Enzyme Assays The activity of free and entrapped γ-GT was measured in 96 well plates using a TECAN Safire absorbance/fluorescence platereader operated in absorbance mode. Solution assays of γ-GT activity were performed by mixing 25 μL of the diluted γ-GT solution (with a concentration equivalent to that in the titania gel matrix) with 25 μL of a solution containing 200 mM of gly-gly and varying concentrations of GPN (0.25-3.35 mM) in each well. The activity of γ-GT in solution was evaluated from the initial rate of product formation over the first 5 min.

Differences in optical transmittance between different titania samples at 410 nm made it impossible to directly measure enzymatic activity in the monolith in situ. Therefore, the activity of titania-entrapped γ-GT was measured by a stop-time assay of the supernatant. Monoliths with or without γ-GT were aged and extensively washed. 100 μL of HEPES/NaOH buffer solution (25 mM, pH 7.0) containing 200 mM gly-gly and varying concentrations of GPN (0.25-3.35 mM) were added to the top of monolith. The reaction proceeded for 45 min, followed by transfer of 50 μL of reaction mixture from the tops of monoliths to another microwell plate where the concentration of product formed was measured immediately at 410 nm.

### Discussion for Example 3

γ-GT Activity Vs. PEO, Glycerol or Isopropanol Levels

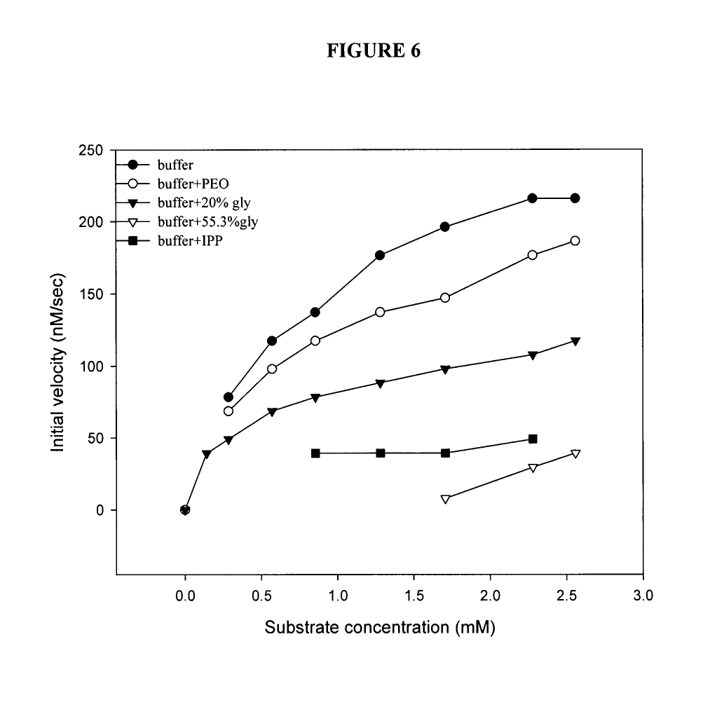

Upon the addition of water to alkyl titanates to initiate hydrolysis, part of the glycerol that initially grafted onto the secondary titanium precursor will be replaced with hydroxyl groups, causing glycerol to be released to the solvent phase. Thus, both glycerol and isopropanol will be present in the sol as hydrolysis processes occur. It was therefore important to establish if the presence of glycerol could serve to protect proteins present in the sol from denaturation by isopropanol. To test this possibility, the enzyme kinetics of γ-GT in solutions containing PEO, glycerol and/or isopropanol at levels similar to those initially present in titania monoliths were examined (FIG. 6). PEO decreased the performance of γ-GT in solution only slightly, as expected, given that PEO has been demonstrated to be a biocompatible polymer.⁸¹ A similar effect was found with chorismate mutase in the presence of PEO 8000.⁸² This is due to the fact that PEO, as a polymeric viscogen, increases the macroviscosity (measured viscosity) of the solution, but has no effect on the diffusion behavior of small molecules, and thus does not alter k_{cat} or k_{cat}/(Kₘ).⁸³

Unlike PEO, the initial rate of substrate turnover by γ-GT was significantly decreased (up to 50%) in the presence of 20% (w/w) glycerol. Glycerol is a commonly used protein stabilizer, and thus it is not likely that glycerol decreases the intrinsic activity of γ-GT. Rather, glycerol is a small-molecule viscogen, and can thus increase both macroviscosity and microviscosity, with the latter decreasing the rates of diffusive processes.^{84,85} This hypothesis was further substantiated by measuring γ-GT kinetics in a solution containing 55.3% (w/w) glycerol. In this case, the rate of substrate turnover is apparently diffusion controlled, as the initial rate of substrate turnover by γ-GT increases linearly with substrate concentration, where the driving force for diffusion is the substrate concentration gradient.

The effect of isopropanol, the hydrolysis product of titanium isopropoxide, on the rate of substrate turnover by γ-GT in solution was also investigated. Isopropanol has a significant deleterious effect on the activity of γ-GT. Furthermore, γ-GT did not follow Michaelis-Menten kinetics in the presence of 17.6% (v/v) isopropanol (the highest possible concentration of isopropanol that would be liberated by the titania sol-gel matrix, based on 100% hydrolysis of the titanium precursor). Isopropanol is a somewhat hydrophilic organic solvent, which has the capability of stripping off essential waters from the γ-GT molecule,⁸⁶ thus it is likely that this compound resulted in partial denaturation of γ-GT. This result suggests that removal of either glycerol or isopropanol from the titania monolith is optimal to minimize the viscosity and the detrimental effects of the alcohol, and thus maximize enzyme activity.

γ-GT Leaching from Titania Monoliths

Given the desirability of to removing glycerol and isopropanol, a washing step was performed prior to activity assays of entrapped γ-GT to minimize the effects of these matrix components on enzyme activity. However, as noted above, titania monoliths prepared with glycerol alone were mesoporous, while those containing PEO were typically macroporous, and thus it was important to assess how the washing step might affect the retention of the entrapped enzyme.

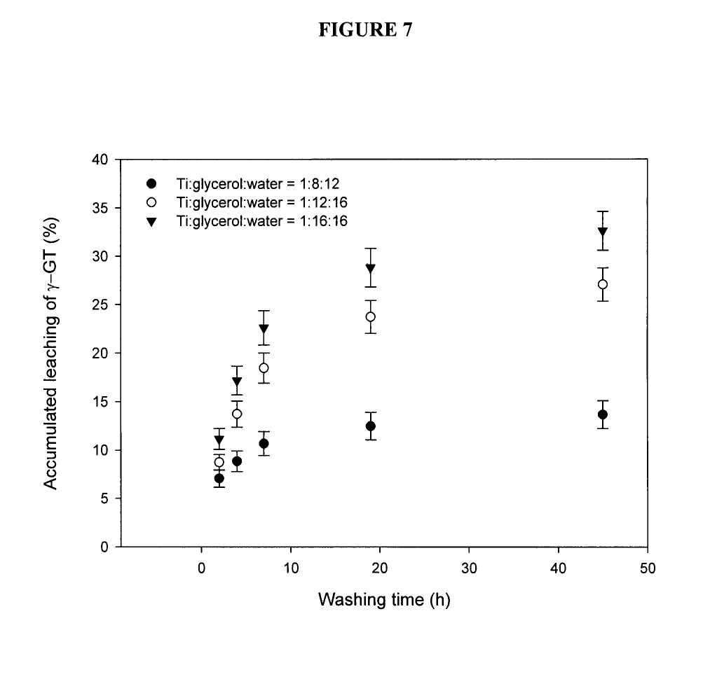

The leaching of γ-GT was tested using titania gel monoliths containing various glycerol and water levels (titanium:glycerol:water molar ratio of 1:16:16; 1:12:16 or 1:8:12), prepared according to composition 1 in FIG. 7. Samples contained 1 wt % of PEO to produce a material which contained both mesopores (−7 nm diameter) and macropores (ca. 700 nm diameter), since such meso/macroporous materials are representative of the materials typically used for fabrication of monolithic bioaffinity columns.⁸⁰ Initial attempts to monitor protein leaching by measurement of the activity of the leached enzyme were unsuccessful, mainly owing to large differences in the solvent composition (i.e., levels of glycerol, PEO and isopropanol) present in the wash solutions obtained from the different titania monoliths. This had a direct effect on enzyme activity, making it impossible to quantify the amount of leached enzyme using activity. For this reason, the fluorescence study of Cy5—labeled γ-GT was used to evaluate the leaching of γ-GT from titania monoliths.

The leaching profile of labeled γ-GT from titania gels containing various levels of glycerol and water is shown in FIG. 7. The leaching profile demonstrated significant losses in entrapped enzyme over the first four washes, which then reached a plateau in subsequent washing cycles. The loss of protein in the initial washing cycles is consistent with γ-GT leaching being driven by diffusion. Interestingly, the accumulated leaching of γ-GT increased from 13.7% to 32.6% when glycerol and water concentration increased, although increased water content was the predominant factor controlling leaching. This is consistent with the expected increases in average pore size as a function of increased water content.⁸⁷ Higher glycerol concentrations may also promote a more porous structure from which a larger proportion of γ-GT may leach upon washing.

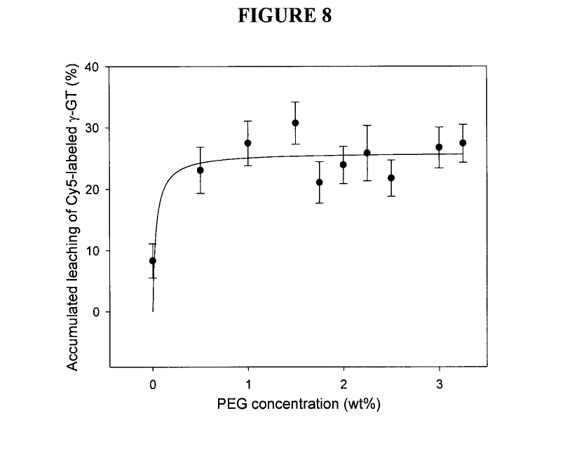

As noted above, the addition of PEO leads to alterations in both meso and macroporosity, with total meso and macropore volume increasing rapidly as PEO concentration is increased from 0-2.5 wt % PEO, and then decreasing slightly as PEO concentration is further increased to 3.25%. To more carefully assess the role of PEO in controlling leaching, titania gel monoliths containing various PEO (10 kDa) concentrations were prepared according to composition 2 in Table 4 with PEO concentrations ranging from 0-3.25 wt % and a total of 20 γg of entrapped protein per gram of gel. The accumulated leaching of γ-GT from titania gels containing various concentrations of PEO is shown in FIG. 8. The leaching of γ-GT increases from 8% to 23% upon the addition of as little as 0.5 wt % of PEO, consistent with the formation of macropores at low levels of PEO. This hypothesis is substantiated by the observed increase in meso and macropore volume at such levels of PEO, and by the opaque appearance of titania monolith in the presence of 0.5 wt % PEO. The overall degree of leaching by diffusion remained essentially constant over the range of 1-3.25 wt % PEO, suggesting the presence of PEO primarily affects macroporosity, but not mesoporosity, as confirmed by the BET data provided in the previous examples. Proteins entrained within mesopores would be expected to be retained during washing. Importantly, even for the macroporous materials a total of ˜70% of the initially added protein remains within the titania matrix when using an initial enzyme loading of 20 μg/g of gel.

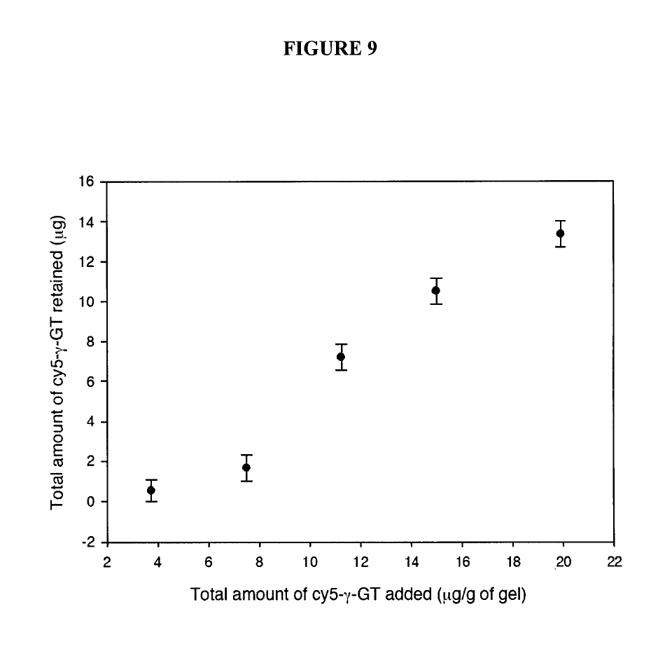

FIG. 9 shows the amount of γ-GT retained in the titania gel matrix (Formulation Table 4) after washing vs. the amount of protein loaded in gel matrix, after accounting for leaching. The amount of γ-GT retained is almost linear with the amount of γ-GT initially entrapped over the concentration range studied, although there appears to be some offset at low enzyme loading that suggests higher leaching in such cases. At higher enzyme loading, up to 70% of the protein is retained, which should allow for the use of this material in applications such as frontal affinity chromatography,⁸⁸ biosensor development, or as immobilized biocatalysts for bioreactors.

### Catalytic Performance of γ-GT in Titania Gel Monolith

The kinetics of substrate turnover by γ-GT entrapped in titania monoliths derived from various molar ratios of the titanium precursor and glycerol were examined (Table 5). All materials were formed with 2 wt % PEO to produce a bimodal meso/macroporous morphology that was appropriate for fabrication of columns and were washed prior to kinetic assays to remove entrained glycerol and isopropanol. In all cases, entrapped γ-GT demonstrated a higher apparent K_{M} than free γ-GT. A similar situation was observed when γ-GT was entrapped in a glycerol-derived silica material.⁸⁹ K_{M} is a parameter reflecting the affinity of the enzyme for its substrate, where higher K_{M} values indicate a lower affinity between the substrate and enzyme. In most cases, K_{M} values of entrapped enzymes increase compared to their solution values, indicating weaker binding of substrates to the enzymes. The entrapment matrix can also impose a barrier for mass transfer of substrate into the matrix, reducing the on-rate of the substrate, k₁ in cases where the reaction is diffusion controlled, and thereby leading to an increase in the K_{M} value (K_{M}=(k₂−k₋₁)/k₁), where k₋₁ is the off-rate of the substrate from the substrate:enzyme complex and k₂ is the rate of conversion of bound substrate to product.

Table 5 also gives the apparent k_{cat} value of γ-GT entrapped in the different titania monoliths. Entrapped γ-GT demonstrated up to 90% of its solution k_{cat} value, indicating that the activity of γ-GT is largely retained in the titania gel. Note that the k_{cat} value was obtained by taking into account the amount of γ-GT that leached upon washing, while assuming that the entrapped enzyme is active and fully accessible. This assumption will likely result in an overestimation of the enzyme concentration and hence an underestimation of k_{cat} (k_{cat}=Vₘₐₓ/[E]) where Vₘₐₓ is the maximum rate of substrate conversion under conditions of substrate saturation at a given enzyme concentration [E]. It is always difficult to obtain the true k_{cat} of entrapped enzyme since it is not typically possible to quantitate occluded protein.⁹⁰ Furthermore, the enzyme may either be active, partially active, active but with altered kinetic behavior, or totally denatured upon entrapment.⁹¹ Thus, both k_{cat} and K_{M} must be treated as apparent values. Even so, the (k_{cat}/K_{M}) for entrapped γ-GT, which reflects the overall catalytic efficiency of the enzyme, was 23-45% of the solution value, suggesting that the entrapped protein retained up to half of its catalytic efficiency upon entrapment. This is significantly better than the catalytic efficiency of γ-GT in silica materials (15% of the solution value⁹²) and far superior to the activity of many other proteins in silica materials, which often show up to 4000-fold lower catalytic efficiency relative to their solution values.⁹⁰ It is encouraging to find that entrapment of the model enzyme γ-GT within titania did not lead to a significant reduction in activity relative to solution.

Knowing that varying the PEO concentration would alter the porosity of gel matrix, it was of interest to examine how the gel properties related to the kinetic properties of entrapped γ-GT). At low PEO concentrations (<1 wt %), PEO has no statistically significant effect on the apparent k_{cat} of entrapped γ-GT (after accounting for leaching). However, the k_{cat} increased by a factor of 2.5 to 7 when the PEO concentration in the titania gel monolith increased from 1 to 2.5 wt %, while further increases in PEO concentration lead to a dramatic decrease in the apparent k_{cat}. As noted above, increasing the PEO concentration initially resulted in the development of large mesopores and significant increases in pore volume. However, further increases in PEO concentration beyond about 2.5 wt % lead to a decrease of both meso and macropore volume, and the disappearance of large mesopores. Indeed, at 2.5% PEO γ-GT exhibited a k_{cat} in titania gel (3.54 s⁻¹) that was comparable to that that in solution (3.92 s⁻¹), demonstrating that entrapment process did not significantly alter the γ-GT kinetic properties, and that mass transfer limitations were minimized in gel with micrometer sized macropores.

The activity of γ-GT entrapped in titania gel (composition 4, Table 4) was measured after storage for the specified time period. The samples were aged in air in the presence of their original pore solvent (i.e., without first washing the matrix) so as to retain the protein stabilizing compound glycerol during aging. However, extensive washing was done just prior to performing the activity assay to remove glycerol, PEO, isopropanol and extractable γ-GT. The activity of γ-GT entrapped in titania remained constant over a period of three weeks, and then slowly decreased to ˜50% of its original activity after 7 weeks under the conditions used for aging. This activity profile vs. aging time is similar to what was reported for γ-GT entrapped in diglycerylsilane (DGS), however it must be noted that the specific activity of γ-GT in titania was ˜3-fold higher than in silica at all aging times.⁹³ Overall, the data suggest that the loss of activity is related to slow denaturation of the entrapped enzyme as the pore solvent evaporates, and indicate that while entrapment does not lead to a loss in initial activity relative to solution, it also does not prevent denaturation during storage. It must be noted that these preliminary results reflect the effects of entrapment for only one enzyme under a single set of storage conditions. However, the ability to retain activity for the entrapped enzyme that is similar to solution indicates the potential for the use of titania as a support for enzyme immobilization.

While the present invention has been described with reference to the above examples, it is to be understood that the invention is not limited to the disclosed examples. To the contrary, the invention is intended to cover various modifications and equivalent arrangements included within the spirit and scope of the appended claims.

TABLE 1
The effect of H₂O/Ti molar ratio on gelation time (hydrolysis of GT16)

| H₂O/Ti molar ratio | Gel time (min) |
|---|---|
| 4 | 2095 |
| 8 | 345 |
| 12 | 180 |
| 16 | 60 |
| 20 | 50 |
| 32 | <1 |

TABLE 2
Varying the content and molecular weight of poly(ethylene oxide)

| | PEO/Ti | Phase | | | Nominal | |
|---|---|---|---|---|---|---|
| | molar ratio | separation | Gelation | | fused | |
| | (PEO | time, tₚₛ | time, t_{g} | t_{g} − | particle size | Shrinkage, |
| Sample | kMW) | (min) | (min) | tₚₛ | (nm) | (dried)^{a} |
| GT16-P1 | 1.0% (10) | 5 | 16 | 11 | 1150 | (cracked) |
| GT16-P0.8 | 0.8% (10) | 5 | 10 | 5 | | 59% |
| GT16-P0.6 | 0.6% (10) | 8 | 28 | 20 | | 65% |
| GT16-P0.5 | 0.5% (10) | 15 | 30 | 15 | 470 | 82% |
| GT16-P0.4 | 0.4% (10) | 20 | 30 | 10 | | 70% |
| GT16-P0.25 | 0.25% (10) | 35 | 41 | 6 | 175 | 90% |
| GT16-P0.125 | 0.125% (10) | 33 | 52 | 19 | 60 | |
| GT16-P0.05- | 0.05% (100) | 50 | 80 | 30 | | |
| 100 | | | | | | |
| GT16- | 0.005% (100) | 30 | 45 | 15 | 115 | |
| P0.005-100 | | | | | | |
| GT16-P5-1 | 5% (1) | NA | 53 | NA | 65 | |
| GT16 | 0% | | | | 70 | 95% |

^{a}Dried at room temperature in air for 60 days.

TABLE 3
The effect of PEO/Ti ratio on pore size distribution as measured by BET and
mercury intrusion porosimetry before and after calcination at 600° C.

| | | | | Mercury Intrusion | | |
|---|---|---|---|---|---|---|
| | Nitrogen Adsorption | | | Porosimetry | | |
| | BET | Total | | Total | Total | Median |
| | surface | pore | Average | surface | intruded | pore |
| | area, | volume, | pore | area, | volume, | size, |
| Sample | m²/g | 10⁻² cc/g | size, nm | m²/g | 10⁻² cc/g | nm |
| Dried at room | | | | | | |
| temperature | | | | | | |
| GT16 | 51.4 | 4.2 | 3.3 | 15.9 | 2.1 | 3.6 |
| GT16-P0.25 | 467.0 | 42.4 | 3.6 | 51.7 | 144.9 | 226.2 |
| GT16-P0.4 | 370.9 | 33.2 | 3.6 | 29.4 | 172.5 | 497.4 |
| GT16-P0.125 | 201.8 | 15.6 | 3.1 | 11.7 | 98.2 | 427.3 |
| GT16-P0.6 | 331.1 | 20.5 | 2.5 | 24.4 | 111.0 | 674.9 |
| GT16-P0.8 | 205.3 | 13.2 | 2.6 | 6.7 | 50.0 | 444.4 |
| GT16-P1 | 12.0 | 2.3 | 7.6 | 3.3 | 45.2 | 690.9 |
| GT16-P0.05-100 | 11.7 | 2.2 | 7.4 | 51.4 | 59.2 | 396.5 |
| GT16-P5-1 | 217.2 | 12.5 | 2.3 | | | |
| Calcined at 600° C. | | | | | | |
| GT16 600° C. | 20.4 | 3.0 | 6.0 | 14.6 | 2.5 | 5.1 |
| GT16-P0.25 600° C. | 21.5 | 6.9 | 12.8 | 11.8 | 37.7 | 145.8 |
| GT16-P0.4 600° C. | 14.2 | 3.8 | 10.6 | 9.3 | 80.3 | 375.1 |
| GT16-P0.125 600° C. | 11.4 | 3.2 | 11.3 | 5.7 | 49.4 | 393.6 |
| GT16-P0.6 600° C. | 5.3 | 1.3 | 9.5 | 4.3 | 49.5 | 482.0 |
| GT16-P0.8 600° C. | 6.3 | 1.4 | 9.0 | 15.5 | 30.5 | 324.6 |
| GT16-P1 600° C. | 7.2 | 1.3 | 7.2 | 2.2 | 28.6 | 533.0 |
| GT16-P0.05-100 600° C. | 17.3 | 2.7 | 6.3 | 5.3 | 29.7 | 336.6 |

TABLE 4
Composition of starting sol used to produce sol-gel derived
titania materials.

| | Titanium | | | |
|---|---|---|---|---|
| | isopropoxide | Glycerol | Water | PEO (10 KDa) |
| Composition | (mmol) | (mmol) | (mmol) | wt % |
| 1 | 1 | 8~16 | 12~16^{a} | 1 |
| 2 | 1 | 12 | 16 | 0~3.25 |
| 3 | 1 | 16 | 16 | 1 |
| 4 | 1 | 12 | 16 | 2 |

^{a}Materials of composition 1 were prepared with a titanium:glycerol:water molar ratio of: 1:16:16; 1:12:16 or 1:8:12.

TABLE 5
Summary of enzyme kinetic properties of γ-GT in solution and when
entrapped into titania materials derived from different sols. All
materials contain 2 wt % PEO and an initial loading of 20 μg
of γ-GT per gram of gel.

| Ti:glycerol:water | | | | k_{cat}/K_{M} |
|---|---|---|---|---|
| ratio | K_{M} (mM) | Vₘₐₓ (μM/sec) | k_{cat} (1/s) | (1/(M · s) |
| Free enzyme | 2.23 | 0.445 | 3.92 | 1.76 * 10³ |
| 1:8:12 | 7.04 | 0.349 | 2.84 | 0.403 * 10³ |
| 1:12:16 | 7.39 | 0.377 | 3.64 | 0.492 * 10³ |
| 1:16:16 | 3.41 | 0.259 | 2.71 | 0.797 * 10³ |

### FULL CITATIONS FOR DOCUMENTS REFERRED TO IN THE SPECIFICATION

1. A very clear discussion of the differences in flow characteristics between different types of silica may be found in Leinweber, F. C.; Lubda, D.; Cabrera, K.; Tallarek, U. Anal. Chem. 2002, 74, 2470.
2. Nogues; J.-L.; Balaban; C.; Moreshead; W. V. U.S. Pat. No. 5,076,980 (to Geltech), 1991.
3. Premstaller, A.; Huber, C.; Oberarcher, H. World Patent Application WO0155713, Method and Apparatus for Separating Polynucleotides Using Monolithic Capillary Columns.
4. Control of pore size in macroporous sol gels: Tanaka, N.; Nagayama, H.; Kobayashi, H.; Ikegami, T.; Hosoya, K.; Ishizuka, N.; Minakuchi, H.; Nakanishi, K.; Cabrera, K.; Lubda, D. J. High Resol. Chromatogr. 2000, 23, 111.
5. (a) Merck column; http://www.chromolith.com/english/services/chromatographie/hplc/chromolith/intro.html. (b) Nakanishi; K.; Soga; N.; Minakuchi; T. PCT publication number WO98/29350 (to Merck Patent GmbH), 1998.
6. a) Hage, D. S. J. Chromatog. B, 1998, 715, 3-38 b) Hage, D. S.; Clin. Chem. 1999, 45, 593-615; c) Weller, M. G. Fresenius J. Anal. Chem. 2000, 366, 635-645; d) Muronetz, V. I.; Sholukh, M.; Korpela, T. J. Biochem. Biophys. Methods 2001, 49, 29-47; e) Baczek, T.; Kaliszan, R. J. Biochem. Biophys. Methods 2001, 49, 83-98; f) Burgess, R. R.; Thompson, N. E. Curr. Opin. Biotechnol. 2002, 13, 304-308.
7. a) Hage, D. S.; Noctor, T. A. G.; Wainer, I. W. J. Chromatogr. A, 1995, 693, 23-32; b) Hofstetter, H.; Hofstetter, O, Schurig, V. J Microcolumn Sep. 1998, 10, 287-291; c) Hofstetter, O.; Lindstrom, H.; Hofstetter, H. Anal. Chem. 2002, 74, 2119-2125. Fitos, I.; Visy, J.; Simonyi, M. J. Biochem. Biophys. Methods 2002, 54, 71-84.
8. a) Hsieh, Y. L.; Wang, H.; Elicone, C.; Mark, J.; Martin, S. A.; Regnier, F. Anal. Chem., 1996, 68, 455-462; b) Wang, C.; Oleschuk, R.; Ouchen, F.; Li, J.; Thibault, P.; Harrison, D. J. Rapid. Commun. Mass. Spectrom. 2000, 14, 1377-1383; c) Wang, S.; Regnier, F. E. J. Chromatogr. A 2001, 913, 429-436; d) Peterson, D. S.; Rohr, T.; Svec, F.; Frechet, J. M. J. J. Proteome Res. 2002, 1, 563-568; e) Peterson, D. S.; Rohr, T.; Svec, F.; Frechet, J. M. J. Anal. Chem. 2002, 74, 4081-4088; f) Slysz, G. W.; Schriemer, D. C. Rapid Commun. Mass Spec. 2003, 17, 1044-1050.
9. Prazeres, D.; Miguel F.; Cabral, J. M. S. in: Multiphase Bioreactor Design, Cabral, J. M. S.; Mota, M.; Tramper, J. (Eds.) Taylor & Francis Ltd., London, UK, 2001, pp. 135-180.
10. a) Schriemer, D. C.; Bundle, D. R.; Li, L.; Hindsgaul, O. Angew. Chem., Int. Ed Engl. 1998, 37, 3383; b) Zhang, B.; Palcic, M. M.; Schriemer, D. C.; Alvarez-Manilla, G.; Pierce, M.; Hindsgaul, O. Anal. Biochem. 2001, 299, 173-182.
11. a) Baynham, M. T.; Patel, S.; Moaddel, R.; Wainer, I. W.; J. Chromatogr. B 2002, 772, 155-161; b) Moaddel, R.; Lu, L.; Baynham, M.; Wainer, I. W. J. Chromatogr. B 2002, 768, 41-53; c) Moaddel, R.; Cloix, J.-F.; Ertem, G.; Wainer, I. W. Pharmaceut. Res. 2002, 19, 104-107; d) Moaddel, R.; Wainer, I. W.; J. Pharmaceut. Biomed. Anal. 2003, 30, 1715-1724.
12. a) Royer, C. A. Prog. Biotechnol. 2002, 19, 17-25; b) Seemann, H.; Winter, R.; Royer, C. A. J. Molec. Biol. 2001, 307, 1091-1102.
13. Gill, I., Chem. Mater. 2001, 13, 3404.
14. Jin, W.; Brennan, J. D. Anal. Chim. Acta 2002, 461, 1.
15. Zheng, L.; Reid, W. R.; Brennan, J. D. Anal. Chem. 1997, 69, 3940.
16. Zheng, L.; Brennan, J. D. Analyst 1998, 123, 1735.
17. Edmiston, P. L.; Wambolt, C. L.; Smith, M. K.; Saavedra, S. S. J. Coll. Int. Sci. 1994, 163, 395.
18. Jordan, J. D.; Dunbar, R. A.; Bright, F. V. Anal. Chem. 1995, 67, 2436.
19. Gottfried, D. S.; Kagan, A.; Hoffman, B. M.; Friedman, J. M. J. Phys. Chem. B 1999, 103, 2803.
20. Doody, M. A.; Baker, G. A.; Pandey, S.; Bright, F. V. Chem. Mater. 2000, 12, 1142.
21. Wambolt, C. L.; Saavedra, S. S. J. Sol-Gel Sci. Tech. 1996, 7, 53.
22. Shen, C.; Kostic, N. M. J. Amer. Chem. Soc. 1997, 119, 1304.
23. a) Braun, S.; Shtelzer, S.; Rappoport, S.; Avnir, D.; Ottolenghi, M. J. Non-Cryst. Solids, 1992, 147, 739; b) Avnir, D.; Braun, S.; Lev, O.; Ottolenghi, M. Chem. Mater. 1994, 6, 1605; c) Wang, R.; Narang, U.; Prasad, P. N.; Bright, F. V. Anal. Chem. 1993, 65, 2671; d) Ellerby, L. M.; Nishida, C. R.; Nishida, F.; Yamanaka, S. A.; Dunn, B.; Valentine, J. S.; Zink, J. I. Science, 1992, 225, 1113; e) Wu, S.; Ellerby, L. M.; Cohan, J. S.; Dunn, B.; El-Sayed, M. A.; Valentine, J. S.; Zink, J. I. Chem. Mater. 1993, 5, 115; f) Dave, B. C.; Soyez, H.; Miller, J. M.; Dunn, B.; Valentine, J. S.; Zink, J. I. Chem. Mater. 1995, 7, 1431; g) Yamanaka, S. A.; Nishida, F.; Ellerby, L. M.; Nishida, C. R.; Dunn, B.; Valentine, J. S.; Zink, J. I. Chem. Mater. 1992, 4, 495; h) Dave, B. C.; Dunn, B.; Valentine, J. S.; Zink, J. I. Anal. Chem. 1994, 66, 1120A; i) Blyth, D. J.; Aylott, J. W.; Richardson, D. J.; Russell, D. A. Analyst 1995, 120, 2725; j) Aylott, J. W.; Richardson, D. J.; Russell, D. A. Analyst 1997, 122, 77; k) Williams, A. K.; Hupp, J. T. J. Am. Chem. Soc. 1998, 120, 4366.
24. a) Braun, S.; Rappoport, S.; Zusman, R.; Avnir, D.; Ottolenghi, M. Mater. Lett. 1990, 10, 1; b) Heichal-Segal, O.; Rappoport, S.; Braun, S.; Biotechnology, 1995, 13, 798; c) Reetz, M. T.; Zonta, A.; Simpelkamp, J.; Biotechnol. Bioengin. 1996, 49, 527; d) Narang, U.; Prasad, P. N.; Bright, F. V.; Kumar, K.; Kumar, N. D.; Malhotra, B. D.; Kamalasanan, M. N.; Chandra, S. Chem. Mater. 1994, 6, 1596; e) Narang, U.; Prasad, P. N.; Bright, F. V.; Ramanathan, K.; Kumar, N. D.; Malhotra, B. D.; Kamalasanan M. N.; Chandra, S. Anal. Chem. 1994, 66, 3139; f) Jordan, J. D.; Dunbar, R. A.; Bright, F. V.; Anal. Chim. Acta 1996, 332, 83; g) Yamanaka, S. A.; Dunn, B.; Valentine, J. S.; Zink, J. I. J. Am. Chem. Soc. 1995, 117, 9095; h) Kauffmann, C.; Mandelbaum, R. T.; J. Biotechnol. 1998, 62, 169.
25. a) Bronshtein, A.; Aharonson, N.; Avnir, D.; Turniansky, A.; Altstein, M. Chem. Mater. 1997, 9, 2632; b) Altstein, M.; Bronshtein, A.; Glattstein, B.; Zeichner, A.; Tamiri, T.; Almong, J. Anal. Chem. 2001, 73, 2461; c) Bronshtein, A.; Aharonson, N.; Turniansky, A.; Altstein, M. Chem. Mater. 2000, 12, 2050; d) Cichna, M.; Knopp, D.; Niessner, R. Anal. Chim. Acta 1997, 339, 241; e) Cichna, M.; Markl, P.; Knopp, D.; Niessner, R. Chem. Mater. 1997, 9, 2640; f) Schedl, M.; Wilharm, G.; Achatz, S.; Kettrup, A.; Niessner, R.; Knopp, D. Anal. Chem. 2001, 73, 5669-5676; g) Spitzer, B.; Cichna, M.; Markl, P.; Sontag, G.; Knopp, D.; Niessner, R. J. Chromatogr. A 2000, 880, 113.
26. Cichna, M. J. Sol-Gel Sci. Technol. 2003, 26, 1159-1164.
27. Zusman R.; Zusman, I. J. Biochem. Biophys. Methods 2001, 49, 175-187.
28. a) Sakai-Kato, K.; Kato, M.; Toyo'oka, T. Anal. Chem. 2002, 74, 2943-2949; b) Kato, M.; Sakai-Kato, K.; Matsumoto, N.; Toyo'oka, T. Anal. Chem. 2002, 74, 1915-1921; c) Sakai-Kato, K.; Kato, M.; Toyo'oka, T. Anal. Biochem. 2002, 308, 278-284; d) Sakai-Kato, K.; Kato, M.; Nakakuki, H.; Toyo'oka, T. J. Pharmaceut. Biomed. Anal. 2003, 31, 299-309; e) Sakai-Kato, K; Kato, M.; Toyo'oka, T. Anal. Chem. 2003, 75, 388-393; f) Kato, M.; Matsumoto, N.; Sakai-Kato, K.; Toyo'oka, T. J. Pharmaceut. Biomed. Anal. 2003, 30, 1845-1850.
29. Macbeath, G.; Schreiber, S. L. Science 2000, 289, 1760.
30. Gill, I.; Ballesteros, A. J. Am. Chem. Soc. 1998, 120, 8587.
31. a) Brook, M. A.; Chen, Y.; Guo, K.; Zhang, Z.; Brennan, J. D. J. Mater. Chem. 2004, 14, 1469-1479. b) Brook, M. A.; Chen, Y.; Guo, K.; Zhang, Z.; Brennan, J. D. J. Sol. Gel. Sci. Technol. 2004, 31, 343-348.
32. a) Kovarik, P.; Hodgson, R. J.; Covey, T.; Brook, M. A.; Brennan, J. D. Anal. Chem. 2005, 77, 3340-3350. b) Cruz-Aguado, J. A.; Chen, Y.; Zhang, Z.; Brook, M. A.; Brennan, J. D. Anal. Chem. 2004, 76, 4182-4188. c) Cruz-Aguado, J. A.; Chen, Y.; Zhang, Z.; Elowe, N. H.; Brook, M. A.; Brennan, J. D. J. Am. Chem. Soc. 2004, 126, 6878-6879.
33 Nakanishi and Brook and Brennan
34. Kawahara, M.; Nakamura, H.; Nakajiam, T. J. Chromat. 1990, 515, 149.
35. Boskovic, S.; Maitland, C. F.; Connolly, J.; Buckley, C. E.; Turney, T. W.; Gee, M. L.; Stevens, G. W.; O'Connor, A. J. Studies in Surface Science and Catalysis 2005, 156 (Nanoporous Materials IV), 717.
36. Brinker, C. J.; Scherer, G. W. Sol-Gel Science, Academic Press: San Diego, 1990.
37. Schubert, U. J. Mater. Chem. 2005, 15, 3701-3715.
38. Jiang, Z.; Zuo, Y. Anal. Chem. 2001, 73, 686.
39. Hoth, D. C.; Rivera, J. G.; Colon, L. A. J. Chromat. A. 2005, 1079, 392.
40. Takemoto, S.; Tsuru, K.; Hayakawa, S.; Osaka, A.; Takashima, S. J. Sol-Gel Sci. Tech. 2001, 21, 97.
41. Matsuda, H.; Nakamura, H.; Nakajima, T. Anal. Sci. 1990, 6, 911.
42. Ikeguchi, Y.; Nakamura, H. Anal. Sci. 2000, 16, 541.
43. Kimura, Y.; Shibasaki, S.; Morisato, K.; Ishizuka, N.; Minakuchi, H.; Nakanishi, K.; Matsuo, M.; Amachi, T.; Ueda, M.; Ueda, K. Anal. Biochem. 2004, 326, 262.
44. Tani. K.; Suzuki, Y. J. Chromat. A. 1996, 722, 129.
45. Chen, X. and Dong, S. Biosens. Bioelectron. 2003, 18, 999.
46. Yu, J.; Ju, H. Anal. Chem. 2002, 74, 3579.
47. Sanchez, C.; Livage, J.; Henry, M.; Babonneau, J. Non-Cryst. Solids. 1988, 100, 65.
48. Sato, S.; Oimatsu, S.; Takahashi, R.; Sodesawa, T.; Nozako, F. Chem. Commun. 1997, 2219.
49. Scherer, C. P.; Pantano, C. G. J. Non-Cryst. Solids. 1986, 82, 246.
50. Doeuff, S.; Henry, M.; Sanchez, C.; Livage, J. J. Non-Cryst. Solids. 1987, 89, 206.
51. Khalil, K. M. S.; Baird, T.; Zaki, M. I.; El-Samahy, A. A.; Awad, A. M. Colloids Surfaces. A: Physicochem. Eng. Aspects 1998, 132, 31.
52. Takenaka, S.; Takahashi, R.; Sato, S.; Sodesawa, T. J. Sol-Gel Sci. Technol. 2000, 19, 711.
53. Ban, T.; Ohya, Y.; Takahashi, Y. J. Sol-Gel Sci. Technol. 2003, 27, 363.
54. Scherer, C. P.; Pantano, C. G. J. Non-Cryst. Solids. 1986, 82, 246.
55. Reeves, R. E.; Mazzeno, L. W. J. Am. Chem. Soc. 1954, 76, 2533.
56. Yamamoto, A.; Kambara, S. J. Am. Chem. Soc. 1959, 81, 2663.
57. Sanchez, C.; Livage, J.; Henry, M.; Babonneau, J. Non-Cryst. Solids. 1988, 100, 65.
58. Judeinstein, P.; Livage, J.; Zarudiansky, A.; Rose, R. Solid State Ionics 1988, 28-30, 1722.
59. Livage, J.; Henry, M.; Sanchez, C. Prog. Solid St. Chem. 1988, 18, 259.
60. Doeuff, S.; Henry, M.; Sanchez, C.; Livage, J. J. Non-Cryst. Solids. 1987, 89, 206.
61. Yao, 1.; Zhang, B. J. Mater. Sci. 1999, 34, 5983.
62. Fujita, K.; Konishi, J.; Nakanishi, K.; Hirao, K. Appl. Phys. Lett. 2004, 85, 5595.
63. Konishi, J.; Fujita, K.; Nakanishi, K.; Hirao, K. Chem. Mater. 2006, 18, 864-866.
64. a) Nakanishi, K.; Komura, H.; Takahashi, R.; Soga, N. Bull. Chem. Soc. Jpn. 1994, 67, 1327-35. b) Nakanishi, K.; Soga, N. Bull. Chem. Soc. Jpn. 1997, 70, 587-92.
65. Nakanishi, K.; Komura, H.; Takahashi, R.; Soga, N. Bull. Chem. Soc. Jpn. 1994, 67, 1327.
66. Nakanishi, K.; Soga, N. Bull. Chem. Soc. Jpn. 1994, 70, 587.
67. a) Kajihara, K.; Nakanishi, K.; Tanaka, K.; Hirao, K.; Soga, N. J. Am. Ceram. Soc. 1998, 81, 2670-2676. b) Kajihara, K.; Yao, T. J. Sol-Gel Sci. Tech. 2000, 19, 219-222.
69. Brinker, C. J.; Scherer, G. W. Sol-Gel Science, Academic Press: San Diego, 1990.
70. Stöver, H. D. H.; Li, K. Dispersion Polymerization, in “Polymeric Materials Encyclopedia”, Salamone, J. C., Ed. CRC Press: Boca Raton, Fla. USA, 1996, Vol. 3, pp. 1900.
71. Brennan, J, et al. PCT Patent Application Publication No. WO 2004/039487, filed Nov. 3, 2003
72. Konishi, J.; Fujita, K.; Nakanishi, K.; Hirao, K. Chem. Mater. 2006, 18, 864-866.
73. a) Brook, M. A.; Chen, Y.; Guo, K.; Zhang, Z.; Brennan, J. D. J. Mater. Chem. 2004, 14, 1469-1479. b) Brook, M. A.; Chen, Y.; Guo, K.; Zhang, Z.; Brennan, J. D. J. Sol. Gel. Sci. Technol. 2004, 31, 343-348
74. a) Kovarik, P.; Hodgson, R. J.; Covey, T.; Brook, M. A.; Brennan, J. D. Anal. Chem. 2005, 77, 3340-3350. b) Cruz-Aguado, J. A.; Chen, Y.; Zhang, Z.; Brook, M. A.; Brennan, J. D. Anal. Chem. 2004, 76, 4182-4188. c) Cruz-Aguado, J. A.; Chen, Y.; Zhang, Z.; Elowe, N. H.; Brook, M. A.; Brennan, J. D. J. Am. Chem. Soc. 2004, 126, 6878-6879.
75. Joint Committee on Powder Diffraction Standards*(JCPDS), sample number is 21-1272, 2601 Park Lane, Swarthmore, Pa. 19081, USA; http://www.icdd.com/
76. Mueller, R.; Kammler, H. K.; Wegner, K.; Pratsinis, S. E. Langmuir 2003, 19, 160-165.
77. a) Hsu, L. S.; Shet, C. Y. Optics Lett. 1985, 10, 638-640. b) Yao, B.; Zhang, L. J. Mat. Sci. 1999, 34, 5983-5987. c) Liu, Y.; Li, J.; Wang, M.; Li, Z.; Liu, H.; He, P.; Yang, X.; Li, J. Crystal Growth Des. 2005, 5, 1643-1649.
78. Nakanishi, K. J. Porous Mater. 1997, 4, 67-112.
79. Soloviev, A.; Tufeu, R.; Sanchez, C.; Kanaev, A. V. J. Phys. Chem. B 2001, 105, 4175-4180.
80. Besanger, T. R.; Hodgson, R. J., Green, J. R. A.; Brennan, J. D. Anal. Chim. Acta, 2006, 564, 106.
81. Brash, J. L. J. Biomater. Sci. Polym. Edn. 2000, 11, 1135.
82. Mattei, P.; Kast, P.; Hilvert, D. Eur. J. Biochem. 1999, 261: 25.
83. Blacklow, S. C.; Raines, R. T.; Lim, W. A.; Zamore, P. D.; Knowles, J. R. Biochem. 1988, 27, 1158.
84. Sierks, M. R.; Sico, C.; Zaw, M. Biotechnol. Prog. 1997, 13, 601.
85. Li, Y.; Feng, L.; Kirsch, J. F. Biochem. 1997, 36, 15477.
86. Halling, P. J. Enzyme Microb. Technol. 1994, 16, 178.
87. Flora K. K.: Dabrowski, M. A.; Musson, S. P.; Brennan, J. D. Can. J. Chem. 1999, 77, 1617.
88. Hodgson, R. J.; Chen, Y.; Zhang, Z.; Tleugabulova, D.; Long, H.; Zhao, X.; Organ, M.; Brook, M. A.; Brennan, J. D. Anal. Chem. 2004, 76, 2780.
89. Besanger, T. R. Chen, Y.; Deisingh, A. K.; Hodgson, R. Jin, W. Mayer, S.; Brook M. A.; Brennan, J. D. Anal. Chem. 2003, 75, 2382.
90. Jin, W.; Brennan, J. D. Anal. Chim. Acta 2002, 461, 1.
91. Brennan, J. D. Appl. Spectrosc. 1999, 53, 106A
92. Besanger, T. R.; Chen, Y.; Deisingh, A. K.; Hodgson, R.; Jin, W.; Mayer, S.; Brook M. A.; Brennan, J. D. Anal. Chem. 2003, 75, 2382.
93. Besanger, T. R.; Hodgson, R. J., Green, J. R. A.; Brennan, J. D. Anal. Chim. Acta, 2006, 564, 106.

1. A method of preparing monolithic biomolecule compatible titania comprising:
(a) combining at least one alkoxytitanium compound with one or more organic polyols under conditions suitable for the reaction of the at least one alkoxytitanium compound with the one or more organic polyols to produce polyol substituted titanium compounds and alcohols, wherein the molar ratio of polyol/alkoxytitanium is greater than about 10; and
(b) hydrolyzing the combination of (a) under conditions suitable for the formation of monolithic titania, wherein said conditions comprise a pH of about 5 to about 9.

2. (canceled)

3. The method according to claim 2, wherein the organic polyol is selected from glycerol, propylene glycol, trimethylene glycol, sorbitol, maltose, trehelose, glucose, sucrose, amylose, pectin, lactose, fructose, dextrose and dextran.

4. (canceled)

5. The method according to claim 1, wherein the polyol substituted titanium compound is diglyceryltitanium or tetraglyceryltitanium.

6. The method according to claim 1, wherein the at least one alkoxytitanium compound is of the formula R₄Ti, where R is any alkoxy group that can be cleaved from titanium under the conditions for performing the method and each R may be the same or different.

7. The method according to claim 6, wherein R is selected from methoxy, ethoxy, isopropoxy and n-butoxy.

8. (canceled)

9. The method according to claim 1, wherein the conditions suitable for the reaction of the at least one alkoxytitanium compound with the one or more organic polyol to produce polyol substituted titanium compounds and alkoxy alcohols include combining, in any order the alkoxytitanium compound(s) and organic polyol(s) neat or in the presence of excess polyol or, optionally in the presence of a solvent, and adjusting the temperature so that it is about 0° C. to about 150° C. for about 1 hour to about 72 hours.

10. An organic polyol titanium compound prepared by combining one or more alkoxy titanium compounds, one or more organic polyols and, optionally, a solvent, under conditions to form the organic polyol titanium compound, wherein the one or more organic polyols is selected from sugar alcohols, sugar acids, saccharides, oligosaccharides and polysaccharides.

11. The compound according to claim 10, wherein the one or more organic polyols is selected from sorbitol, maltose, trehelose, glucose, sucrose, amylose, pectin, lactose, fructose, dextrose or dextran.

12. The compound according to claim 10, wherein the conditions to form the organic polyol titanium compound comprise combining, in any order, the alkoxytitanium compound(s) and organic polyol(s) neat or in the presence of excess polyol or, optionally in the presence of a solvent, and adjusting the temperature so that it is about 0° C. to about 150° C.

13. A method for preparing macroporous monolithic titania comprising:
(a) combining at least one alkoxytitanium compound with one or more organic polyols under conditions suitable for the reaction of the at least one alkoxytitanium compound with the one or more organic polyol to produce polyol substituted titanium compounds and alcohols; and
(b) hydrolyzing the combination of (a) in the presence of one or more water soluble polymers under conditions suitable for phase separation to occur before gelation and for the formation of monolithic titania.

14. (canceled)

15. The method according to claim 14, wherein the organic polyol is selected from glycerol, propylene glycol, trimethylene glycol, sorbitol, maltose, trehelose, glucose, sucrose, amylose, pectin, lactose, fructose, dextrose and dextran.

16. (canceled)

17. The method according to claim 13, wherein the polyol substituted titanium compound is diglyceryltitanium or tetraglyceryltitanium.

18. The method according to claim 13, wherein the at least one alkoxytitanium compound is of the formula R₄Ti, where R is any alkoxy group that can be cleaved from titanium under the conditions for performing the method and each R may be the same or different.

19. The method according to claim 18, wherein R is selected from methoxy, ethoxy, isopropoxy and n-butoxy.

20. (canceled)

21. The method according to claim 13, wherein the one or more water soluble polymers are selected from polyethers, poly alcohols, polysaccharides, poly(vinyl pyridine), polyacids, and polyacrylamides.

22. The method according to claim 21, wherein the one or more water soluble polymers are selected from PEO, PEG, polyNIPAM and PAM.

23. The method according to claim 22, wherein the water soluble polymer is PEO.

24. (canceled)

25. The method according to claim 24, wherein the MW of the PEO is between about 10,000 and 100,000 g/mol.

26. The method according to claim 25, wherein the PEO is used at a weight percent of about 0.1 wt % to about 1% for 10,000 g/mol PEO, and at a weight percent of about 0.001 wt % to about 0.1 wt % for 100,000 g/mol MW PEO.

27. The method according to claim 13, wherein the rate of gelation is controlled by addition of polyols, water concentration and pH.

28. The method according to claim 1, further comprising hydrolyzing and condensing the polyol substituted titanium compounds in the presence of one or more biomolecules under conditions suitable for the formation of monolithic titania.

29. A titania material prepared using a method according to claim 1.

30. (canceled)

31. A chromatographic column, microarray or bioaffinity column comprising a material according to claim 29.

32. The method according to claim 13, further comprising hydrolyzing and condensing the polyol substituted titanium compounds in the presence of one or more biomolecules under conditions suitable for the formation of monolithic titania.

33. A titania material prepared using a method according to claim 13.

34. A chromatographic column, microarray or bioaffinity column comprising a material according to claim 33.
